SEQUENCE LISTING (3 sequences)

SEQ ID NO: 1 - DNA, 2271 bp, Bos taurus
gcttgccatgcccgtgaggggctgcccggcacgccagccactcgcacagagagtgcccgagcctgcggtcctcatgtcaggtgacacgggccccccaaagcagggagggaccagatatggctccatctccagcccacccagtccagagccacagcaagcacctcccggagggacctacctaagtgagaagatccccattccggatacagaatcgggtacattcagcctgaggaagctgtgggccttcacggggcctggattcctcatgagcatcgcattcctggacccaggaaacattgagtcggatcttcaggctggggctgtggctggattcaaactgctctgggtgctgctgtgggccacagtgttgggcttgctttgccagcgactggctgcccggctgggcgtggtgacaggcaaggacttgggcgaggtctgccatctctactaccctaaggtgccccgcattctcctctggctgaccatcgagctagccatcgtgggctcagacatgcaggaagtcattggcacagctattgcattcagtctgctctccgccggacgaatcccactctggggtggtgtcctcatcaccgtcgtggacactttcttcttcctcttcctcgataactacgggttgcggaagctggaagccttttttggatttcttattaccataatggccttgaccttcggctatgagtacgtggtggctcagcctgctcagggagcattgcttcagggcctgttcctgccctcgtgcccaggctgtggccagcccgagctgctgcaagccgtgggcatcattggcgccatcatcatgccccacaacatctacctgcattcctccctggtcaagtctcgagaggtagaccggtcccggcgggcggacatccgagaggccaacatgtacttcctgattgaagccaccatcgccctgtctgtctccttcctcatcaacctgtttgtcatggctgtctttgggcaagccttctacaagcaaaccaaccaggctgcgttcaacatctgtgccgacagcagcctccacgactacgcgccgatctttcccaggaacaacctgaccgtggcagtggacatttaccaaggaggcgtgatcctgggctgcctctttggtcctccagccctgtacatctgggccgtgggtctcctggctgctgggcagagctccaccatgaccggcacctacgcgggacagtttgtgatggagggcttcctgaagctgcggtggtcacgcttcgcccgagtcctgctcactcgctcctgcgccatcctgcccactgtgctcctggctgtcttcagggacttgcgggacctgtcaggcctcaacgacctgctcaatgtgctgcagagcctgctgcttcccttcgctgtgctgcccatcctcaccttcaccagcatgcccgccctgatgcaggagtttgccaatggcctggtgagcaaagttatcacttcctccatcatggtgctggtctgcgccgtcaacctttacttcgtgatcagctacttgcccagcctcccccaccctgcctacttcagccttgtagcactgctggccgcagcctacctgggcctcaccacttacctggtctggacctgtctcatcacccagggagccactcttctggcccacagttcccaccaacgcttcctgtatgggcttcctgaagaggatcaggagaaggggaggacctcgggatgagctcccaccagggcctggccacgggtggaatgagtgggcacagtggcctgtcagacaagggtgtgtgtgtgtgtgtgtgtgtgtatgtgtgtgaaggcagcaagacagacagggagttctggaagctggccaacgtgagttccagagggacctgtgtgtgtgtgacacactggcctgccagacaagggtgtgtgtgtgtgtgtgtgtgtgtgtgcatgcacagcaagacggagagggagttctggaaggcagccaacgtgagttccatagggacctgctatttcctagctcagatctcagtgttcttgactataaaatggggacacctaccttggagtggttgtaaataagacacttgaacgcagagcctagcacttcagatttaaaaacaaaagaatcataattccaaaagttactgagcactatcacaggagtgacctgacagacccacccagtctagggtgggacccaggctccaaactgatttaaaataagagtctgaaaatgctaaataaatgctgttgtgcttagtccccgagaaaaaaaaaaaa

SEQ ID NO: 2 - protein, 548 aa, Bos taurus
MSGDTGPPKQGGTRYGSISSPPSPEPQQAPPGGTYLSEKIPIPDTESGTFSLRKLWAFTGPGFLMSIAFLDPGNIESDLQAGAVAGFKLLWVLLWATVLGLLCQRLAARLGVVTGKDLGEVCHLYYPKVPRILLWLTIELAIVGSDMQEVIGTAIAFSLLSAGRIPLWGGVLITVVDTFFFLFLDNYGLRKLEAFFGFLITIMALTFGYEYVVAQPAQGALLQGLFLPSCPGCGQPELLQAVGIIGAIIMPHNIYLHSSLVKSREVDRSRRADIREANMYFLIEATIALSVSFLINLFVMAVFGQAFYKQTNQAAFNICADSSLHDYAPIFPRNNLTVAVDIYQGGVILGCLFGPPALYIWAVGLLAAGQSSTMTGTYAGQFVMEGFLKLRWSRFARVLLTRSCAILPTVLLAVFRDLRDLSGLNDLLNVLQSLLLPFAVLPILTFTSMPALMQEFANGLVSKVITSSIMVLVCAVNLYFVISYLPSLPHPAYFSLVALLAAAYLGLTTYLVWTCLITQGATLLAHSSHQRFLYGLPEEDQEKGRTSG

SEQ ID NO: 3 - DNA, 155 bp, Bos taurus
  modified_base (35)..(129): N = A, C, G or T/U
gggtgtgtgtgtgtgtgtgtgtgtgtatgtgtgtnnnnnnnnnnnnnnnnnnnnnnnnnnnnnnnnnnnnnnnnnnnnnnnnnnnnnnnnnnnnngtgtgtgtgtnnnnnnnnnnnnnnnnnnnnnnnngtgtgtgtgtgtgtgtgtgtgtgtgt

(19) United States
(12) Patent Application Publication
(10) Pub. No.: US 2003/0150002 A1
(43) Pub. Date: Aug. 7, 2003

(54) Cloning bovines by nuclear transplantation

(76) Inventors: Mark E. Westhusin, College Station, TX (US); L. Garry Adams, College Station, TX (US); Doris Hunter, College Station, TX (US); Joe Wayne Templeton, College Station, TX (US); Taeyoung Shin, College Station, TX (US)

Correspondence Address: Gina N. Shishima, Fulbright & Jaworski L.L.P., Suite 2400, 600 Congress Avenue, Austin, TX, 78701, US

(21) Appl. No.: 10/017,902

(22) Filed: Dec. 14, 2001

Related U.S. Application Data
(60) Provisional application No. 60/256,576, filed on Dec. 18, 2000.
(60) Provisional application No. 60/255,860, filed on Dec. 15, 2000.

(51) Int. Cl. A01K 67/027; A01N 1/02; C12N 15/85

(52) U.S. Cl. 800/15; 435/2; 435/455

(57) ABSTRACT

Nuclear transfer methods and techniques involving cryopreserved bovine somatic cells, nuclei, or nuclear DNA, and the products thereof, are disclosed. These methods employ an enucleated oocyte as a recipient, which is fused with a donor nuclear genome from a cryopreserved bovine somatic cell to form a couplet. In addition, the a cloned bull exhibiting disease resistance is disclosed.

The present application claims priority to U.S. Provisional Application No. 60,256,576, filed on Dec. 18, 2000 and U.S. Provisional Application No. 60/255,860 filed on Dec. 15, 2000, both of which are incorporated by reference.

### BACKGROUND OF THE INVENTION

1. Field of the Invention

The present invention relates generally to the fields of molecular biology, embryology and cloning. More particularly, it concerns techniques for and the animals produced by the cloning of bovines.

2. Description of Related Art

The reconstruction of mammalian embryos by the transfer of a nucleus from a donor embryo to an enucleated oocyte or one cell zygote allows the production of genetically identical individuals. This has clear advantages both for research (i.e. as biological controls) and in commercial applications (i.e. multiplication of genetically valuable livestock, uniformity of meat products, animal management). To date, cloned sheep, swine, cattle, and mice have been produced by nuclear transplantation using somatic cells obtained from adult animals (WO9937143A2, EP930009A1, WO9934669A1, WO9901164A1, U.S. Pat. No. 5,945,577, which are herein expressly incorporated by reference). To date, these procedures have been largely restricted to genetic materials derived from living donors. Nevertheless, since the prospect of cloning was conceived, it has always been a desire to clone animals from cryopreserved materials, which allows for the recapture of desirable traits potentially lost when an animal, breed or species expired or became extinct.

Currently, livestock animals in the United States are generally bred to enhance commercially valuable characteristics, such as marbling and tenderness in beef cattle and milk volume and milk protein and fat concentration in dairy cattle. Animals exhibiting these characteristics are generally selected over animals comprising a more disease resistant phenotype due to the assumption that the North American climate and the quality of husbandry practices and veterinary care reduce potential losses from disease. Nevertheless, diseases such as ruminant brucellosis, tuberculosis, paratuberculosis and salmonellosis cause an estimated $250,000,000 loss annually to the United States beef and dairy industry. These diseases cause significant losses in livestock industries despite widespread application of antimicrobials, vaccination, isolation and quarantine, test and slaughter, or a combination of these. The lack of success in eradicating infectious diseases of animals using these approaches indicates a need for a different strategy, such as the identification within commercially valuable breeds of animals exhibiting disease resistance or the engineering of such breeds to exhibit greater disease resistance.

Further, tuberculosis especially is a health threat to all ungulates including rare and endangered mammals. These are diseases for which the usual eradication programs have been long-term, expensive, and somewhat unsuccessful. For example, bovine tuberculosis was thought to be a disease of antiquity in 1970 but has re-emerged as an endemic disease in the El Paso, Tex. dairy herds. Outbreaks of bovine tuberculosis have been reported in the past 5 years in California, Idaho, Indiana, Louisiana, Missouri, Montana, Nebraska, New Mexico, New York, North Carolina, Pennsylvania, South Carolina, Texas, Wisconsin, and Virginia (Essey and Koller 1994; and Essey M. A. 1991).

Further, each of these specific diseases are zoonotic diseases which continually threaten the U.S. population. The benefit of cattle naturally resistant to these diseases, and other diseases, would be a key component of the preharvest pathogen reduction programs like the National Hazard Analysis Critical Control Point (HACCP) program proposed for farm use (Pierson, M. D. and Corlett, D. A., 1992; and Vanderzant, C., 1985). Further, it is desired that the approach used to control these diseases use natural resistance since it is environmentally compatible.

The only method currently available for the detection of artiodactyla resistant to brucellosis or tuberculosis is by a potent in vivo challenge with virulent Brucella abortus, Salmonella dublin, Mycobacterium paratuberculosis, or Mycobacterium bovis (Templeton and Adams 1996). Unfortunately for this assay the tested ungulates have to be euthanized in order to culture the specific pathogen. Males challenged with B. abortus or M. bovis must be necropsied and cultured to determine if the bacterium has been cleared (resistant) or persists (susceptible). Nonpregnant females challenged with M. bovis must be necropsied and cultured to determine resistance or susceptibility. Although the gametes from both males and females can be stored frozen and used in a breeding-selection program to produce naturally resistant progeny with some success, this is both extremely expensive and inefficient. The viability of frozen gametes and embryos is variable, and a much lower birth rate occurs than with natural matings. Additionally, the breeding-selection program would be based on phenotypic selection (so-called mass selection), which is not as efficient as determining genotypes and selecting resistance associated with genetic sequences directly. (See, for example, Martin et al. 1994; and Dietrich et al. 1986).

The present invention provides a method to preserve those characteristics of species, breeds and animals exhibiting advantageous genotypic characteristics that might otherwise be lost. In particular, a bovine has been cloned from a deceased bull that possessed advantageous characteristics.

### SUMMARY OF THE INVENTION

The instant invention sets forth techniques and methods for the cloning of a mammal from cryopreserved tissue based upon nuclear transplantation. Embodiments of the invention encompass the use of cryopreserved material in a method to exactly replicate and transfer the genome of an organism from a somatic cell to produce an animal having an identical nuclear DNA complement, i.e., a clone. While techniques for cloning bovines are known, the instant invention sets forth techniques necessary to achieve cloning by nuclear transfer of cryopreserved material to an enucleated donor cell in bovines.

The instant invention sets forth methods for transferring a bovine nuclear genome from a somatic cell into a recipient cell to produce bovines that are genetically identical to the somatic cell. While the technique of bovine cloning is known, the instant invention discloses the method of cloning from cryopreserved material, including material cryopreserved for well over a decade. This involves achieving replication of the cryopreserved nuclear genome in a cell from which the genome was not derived. In some embodiments, these methods include contacting an enucleated oocyte from a donor organism with a composition that includes nuclear DNA from a cryopreserved bovine somatic cell, fusing the oocyte and the composition containing nuclear DNA from a somatic cell to form a cybrid, and transferring the cybrid into the reproductive tract of a cow. “Contacting” refers to the coming together, touching, associating, or juxtapositioning of one or more objects, which in the context of the present invention includes cells, nuclei, and genetic material.

For the purpose of the instant invention, “cybrid” refers to a cellular unit or structure that is composed of all or part of an oocyte cell and the nuclear DNA of a somatic cell and that is capable of undergoing cell division. The term “cybrid” encompasses the terms “fused oocyte,” “NT (nuclear transfer or tranplantation) embryo,” and “cloned embryo.” It is contemplated that the nuclear DNA may be in a nucleus and/or that the nucleus is contained in a somatic cell. An “embryo” refers to the developmental stage following the first cleavage of a diploid zygotic cell and preceding the fetal stage. An “enucleated” oocyte refers to an oocyte lacking all or part of the nucleus, including nuclear DNA. “Somatic cell” refers to any cell that is not a sex cell; a sex cell is haploid, while a somatic cell is not haploid. This method facilitates the production in a surrogate mother of an offspring whose mitochondrial DNA is derived mainly from one cell while its genomic DNA is derived from another cell. The product of this process is, therefore, a viable bovine, which is included in the invention. For the purpose of the instant invention, a “cloned bovid” or “cloned bovine” refers to a bovine animal originally derived from the transfer of a nuclear genome from a bovine somatic cell into a recipient cell, as opposed to being derived from the union of two sex cells.

For the purpose of the instant application it is contemplated that the term “cryopreserve” refers to preservation by freezing. Thus, cells, organs, tissue, organisms or other biological material that is preserved by freezing are contemplated to constitute cryopreserved material.

It is contemplated that “nuclear DNA” refers to that portion of the genome that is located in the nucleus, contrary to “mitochondrial DNA,” which refers to that portion of the genome located in the mitochondria of a cell. “Nuclear genome” similarly refers to that portion of the genome located in the nucleus, while mitochondrial genome refers to the DNA located in the mitochondria. The nuclear genome may contain heterologous DNA sequences, for example, transgenic DNA sequences. “Heterologous” sequences refers to nucleic acid that is derived from a different source, i.e., organism, than the remainder of the DNA or to DNA sequences found in a location in the nuclear genome where it is not usually found.

In other embodiments, the methods further comprise a step of activating in vitro either before during or after fusion the cybrid or the oocyte. Activation may be achieved through the application of an electrical pulse or, for example through the administration of a chemical compound or compounds, or by physical manipulation.

In some embodiments of the present invention, the cybrid is transferred into the reproductive tract of a female cow immediately or at least before cell division occurs. While in other embodiments, the cybrid is incubated under conditions to promote cell division. The cybrid may undergo 1, 2, 3, 4, 5, 6, 7, 8, 9, 10, or more cell divisions, up to and including the development to the morula or blastocyst stage before being transferred. For example, the cybrid may undergo at least two cell divisions before it is placed in a female surrogate.

Methods of the present invention do not require that the oocyte be isolated from a bovine, though it is contemplated that bovine oocytes or bovine oocytes may be employed. It is further contemplated that the composition may contain any aqueous material such as media and/or cellular material, such as all or part of a nucleus, or all or part of a cell. In some embodiments, the oocyte and the nuclear DNA, nucleus containing nuclear DNA, or somatic cell containing a nucleus containing nuclear DNA are from the same bovine species and in a further embodiment, the oocyte and nucleus are from the same breed. The present invention also includes methods and products of the methods in which an oocyte from a female is used for nuclear transfer involving a somatic cell from the same female animal. Throughout this application, the phrase “nucleus/cell” is intended to refer to a nucleus and/or a cell. It is further contemplated that either nuclear genomic material or a nucleus containing nuclear genomic material may be used interchangeably instead of a somatic cell comprising a nucleus in any of the methods described herein.

In some embodiments of the invention, a nucleus is isolated from a cell, for example, by physical manipulation, before coming in contact with an enucleated oocyte. A person of ordinary skill would recognize a variety of means of inserting a nucleus or the nuclear material from a cell into an enucleated oocyte. Alternatively, a cell containing a nucleus may be contacted and fused with an enucleated oocyte. In some embodiments of the instant invention, the oocyte and the nucleus/cell are fused by applying an electrical pulse. In specific embodiments of the invention, incubating the oocyte and the nucleus/cell in fusion media may further facilitate fusion. In further aspects utilizing the instant methods, the fused oocyte and nucleus/cell, i.e., the cybrid, is cultured in vitro for at least 24 hours prior to transfer. It vitro culture may alternatively be carried out until the embryo reaches at least the 4-8 cell stage or later.

In alternate aspects of the invention, it is contemplated that oocytes could be isolated from a number of organisms or species. In some embodiments of the invention, the donor oocyte is isolated from a bovine or bovine. It is contemplated that the oocyte and nucleus may both be derived from a bovine, isolated from the same bovine species and even, potentially, from the same breed, or animal. Nevertheless, in alternate aspects of the instant invention, the donor oocyte may be from isolated from a species other than a bovine. In alternative embodiments, the oocyte may be either bovine, murine, lapine, feline, caprine, porcine, ovine, equine, or other species that a person of ordinary skill would view as appropriate.

The synchronization of estrus and the developmental stage of the cybrid is an aspect of the instant invention. In some aspects of the claimed invention, the estrous cycle of the surrogate female cow in which the cybrid is transferred is synchronized with the estrous cycle of the oocyte donor female. Two females may be considered synchronized if the oocyte donated by one female is capable of being fused with a nucleus/cell and successfully transferred and implanted in a second female. Thus, it is contemplated that there is a time window that encompasses both females, and that such window may be within 1, 2, 3, or more days of synchrony.

The methods of the invention may involve an oocyte or cybrid that is frozen. In these cases, synchrony between the female donating the oocyte and the female acting as the surrogate is less significant. In other embodiments, synchronization of the surrogate female and the developmental stage of the cybrid is involved. For example, a surrogate female may be 0, 1, 2, 3, 4, 5, 6, 7 or more days or 1, 2 or more weeks post estrus and the cybrid, at a corresponding stage of development such as the 1-, 2-, 4-, 8-, 16-, 32-, 64-, 128-, 236-cell or up to morula or blastocyst stage, may be transferred during those days or weeks.

Hormones, including progesterone, estrogen, or a combination thereof, may be administered to females involved in the methods of the present invention. Females donating oocytes may be given hormones prior to oocyte retrieval. Surrogate females may be given hormones before or during pregnancy.

In some embodiments, the donor oocyte is employed when the oocyte is in metaphase II. Certain aspects of the instant invention require the isolation of an oocyte from a donor. In an alternate embodiment of the instant invention, the isolated oocyte is cultured in vitro prior to enucleation. This culturing period may encompass 1, 2, 3, 4, 5, 6, 7, 8, 9, 10, 11, 12, 13, 14, 15, 16, 17, 18, 19, 20, 21, 22, 23, or 24 hours, as well as 1, 2, 3, 4, 5, 6, or 7 or more days.

In the methods and compositions of the claimed invention, nuclear DNA may contain heterologous DNA sequences, such as a transgene. A transgene connotes a gene that has been transferred from one organism or species to another by genetic engineering.

The instant invention specifically contemplates the production of viable offspring from the creation of an embryo through nuclear transplantation. Therefore, embodiments of the instant invention encompass a bovine whose nuclear genome is identical to a single parent, wherein the parent is multicellular. The bovine animals of the present invention are cloned bovines. A cloned bovine may have cells whose nuclear genome is identical to the nuclear genome of a cell not obtained from the cloned bovine. It may differ from naturally-occurring identical twins because some of its mitochondrial genome may be derived from a cell source that differs from the cell source of its nuclear genome. The mitochondrial genome of a cloned bovine may be derived from one or more sources, including a recipient oocyte and/or the somatic cell from which the nuclear genome was derived.

The term “parent” is used to refer to a provider of nuclear genetic material. The word “single parent” refers to a sole provider of nuclear genetic material. The term “multicellular” indicates an entity composed of more than a single cell, and thus, morulas, blastocyst, embryos, fetuses, and more developed forms of an organism are included. Therefore, the parent may be a morula, blastocyst, embryo, fetus, and up to and including an adult animal, which refers to an animal that has reached sexual maturity. An animal that is more developed than a fetus, but not yet an adult may also be a single parent, with respect to the offspring bovine or cloned bovine of the present invention.

Methods of obtaining such an offspring bovine are disclosed herein. The cloned bovines of the present invention may be produced using any of these methods. “Offspring” or “progeny” refer to any and all subsequent generations of an organism. “Immediate offspring” and “immediate progeny” mean the next generation. The term “parent” refers to the immediate previous generation (may be one or two parents) that gave rise to an organism; “grandparent” refers to the ancestral generation once removed from an organism. The term “ancestor” refers to any and all predecessor generations of an organism. Thus, an ancestor may have given rise to 1, 2, 3,4, 5, 6, 7, 8, 9, 10, 11, 12, 13, 14, 15, 16, 17, 18, 19, 20, 21, 22, 23, 24, 25, or more generations of progeny.

It is contemplated that animals produced by the disclosed methods will be reproductively viable. Therefore, an embodiment of the invention includes the immediate progeny produced by a mating between the bovine produced by the disclosed methods and a second bovine, as well progeny produced by subsequent matings involving any and all generations (progeny) of the immediate progeny.

The instant invention is further envisioned to specifically encompass a bovine whose nuclear genome is identical to a single parent or ancestor, wherein the single parent or ancestor has a polymorphic NRAMP1 gene. In a specific embodiment of the invention, the polymorphic Nramp1 gene confers or evidences disease resistance, for example disease resistance to a disease caused by an intracellular pathogen. Examples of intracellular pathogens contemplated in the context of the instant invention, are, for example: brucellosis, tuberculosis, paratuberculosis or salmonellosis. In a further embodiment of the invention, the polymorphism is found within the 3′ UTR. The polymorphism may be located, for example at nucleotides 1782 or 1789 or both. In a further aspect of the invention, the bovine claimed has a genome substantially identical to that of the genome of ATCC# MXXM cells.

In an additional embodiment, the claimed bovine is produced by a method comprising contacting an enucleated oocyte with a composition of nuclear DNA from a bovine somatic cell derived from a cell line comprising a polymorphic Nramp1 gene. The composition is fused with an enucleated oocyte to form a cybrid and the cybrid transferred into the reproductive tract of a cow. In a specific embodiment of the invention, the polymorphic NRAMP1 gene confers disease resistance, for example disease resistance to a disease caused by an intracellular pathogen. Examples of intracellular pathogens contemplated in the context of the instant invention, are, for example: brucellosis, tuberculosis, paratuberculosis or salmonellosis. In a further embodiment of the invention, the polymorphism is found within the 3′ UTR. The polymorphism may be located, for example at nucleotides 1782 or 1789 or both. In a further aspect of the invention, the bovine claimed has a genome substantially identical to that of the genome of ATCC# MXXM cells or a bovine made from such a cell or cell lines.

The definitions are included in an effort to assist in the proper construction of terms used within the application. As used herein the specification, “a” or “an” may mean one or more. As used herein in the claim(s), when used in conjunction with the word “comprising”, the words “a” or “an” may mean one or more than one. As used herein “another” may mean at least a second or more.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### DESCRIPTION OF ILLUSTRATIVE EMBODIMENTS

The technology described herein represents methods for and products of a strategy for cloning mammals, including bovines, from cryopreserved or freeze dried material, as well as methods for and products of a strategy for cloning a bull with desirable characteristics. Methods for cloning bovines and other ungulates are well known in the art (See, for example U.S. Pat. Nos. 6,011,197, 6,107,543, and 6,147,276 herein expressly incorporated by reference). The general approach disclosed herein involves nuclear transplantation whereby cell nuclei derived from cryopreserved or freeze dried embryonic, fetal, juvenile, or adult cells (nucleus donor cells) are transplanted into enucleated oocytes. Oocytes containing the transplanted nucleus are then artificially activated so to initiate embryonic development. The transplanted nuclei are reprogrammed to direct normal embryonic development. In the context of the instant invention, “reprogrammed nucleus” denotes a nucleus that is capable of directing embryogenesis and further embryonic development. The resulting cybrids can be transferred into surrogate females to produce cloned fetuses and offspring. The cybrids can also be used to produce chimeric embryos, fetuses and/or offspring. In addition, cybrids may be used to derive cells for another round of cloning. Nucleus donor cells may be cultured so to increase the number of cells available for nuclear transplantation, and frozen and thawed prior to use. Under the proper conditions, nucleus donor cells can be collected from deceased animals, cultured, frozen, thawed and used as nucleus donors for nuclear transplantation. Donor cells used for nuclear transplantation can be frozen and stored in liquid nitrogen for many years prior to thawing and using as nucleus donors for cloning by nuclear transplantation. It is specifically contemplated that other methods of DNA preservation, such as, for example, freeze drying, may function in the context of the instant invention to provide the necessary genetic material to produce a cybrid. The only prerequisite is that the preservation process maintain the DNA in a substantially intact condition such that it is capable of being reprogrammed following nuclear transplantation so as to direct embryonic development. Nucleus donor cells can also be genetically modified prior to utilization for nuclear transplantation therefore providing a method for producing genetically engineered animals. For example, DNA sequences may be added or deleted (e.g., knockout).

This invention further represents the successful cloning of a black angus bull exhibiting marked disease resistance, specifically to intracellular pathogens, such as, for example the causative agents of brucellosis, tuberculosis, paratuberculosis and salmonellosis. This disclosure exhibits that clones may be successfully derived from frozen or cryopreserved materials such that desirable genetic traits from organisms, breeds or species thought lost may be renewed.

A. Preparation and Collection of Oocytes

It is currently believed that unfertilized oocytes matured to the metaphase II stage of meiosis are the most appropriate recipient oocytes for utilization in nuclear transfer to clone cows. Nevertheless, it is specifically contemplated that oocytes at others stages of meiosis may also serve as recipient oocytes for reprogramming somatic cell nuclei so to direct normal embryonic development of cloned animals produced using the disclosed methods. The following describes methods for obtaining metaphase II oocytes from cows that can be used as recipient ova for nuclear transfer. However, any unfertilized oocyte that has had its nucleus removed (see below) and is capable of reprogramming somatic cell nuclei and directing normal embryonic development may be used for cloning cattle. These might include oocytes at various stages of meiosis and even include oocytes obtained from other animal species.

In vitro maturation has been implemented for obtaining bovine oocytes for use as recipient ova for nuclear transfer, as large numbers of unfertilized oocytes are obtainable using this method. It is contemplated that bovine oocytes may also be matured in vivo. A variety of techniques may be employed to produce in vitro matured bovine oocytes. The following represents one example of methods that may be employed. Bovine ovaries are collected from a slaughterhouse and transported to the laboratory where immature oocytes are aspirated from follicles. Follicular aspirates are then examined under a stereo microscope and the oocytes recovered and placed into fresh buffer. Oocytes are then placed into culture wells containing 250 μl of maturation medium at 39° C. in an atmosphere of 5% CO₂ and air. The time required for in vitro maturation may vary, but is normally approximately 24 to 96 hours. It is contemplated that oocytes may be matured in vitro for 1, 3, 6, 8, 12, 24, 36, 48, 60, 72, 84, 96, 108, 120, 132, 144, 156, 168, 180, 192, 204, or more hours.

Alternatively, bovine oocytes that have been matured in vivo may be obtained and utilized as recipient oocytes for nuclear transfer to clone bovines. A potential advantage of using these oocytes is they have undergone meiotic maturation under more natural conditions and therefore are expected to function more efficiently in terms of reprogramming cell nuclei and directing normal development of cloned bovine embryos. The disadvantage of in vivo matured oocytes is they must be collected from live animals using surgical procedures or post mortem. Also the number of in vivo matured oocytes that can be obtained for utilization for nuclear transfer is much more limited when compared to the number of oocytes available when employing methods for in vitro maturation.

Collection of oocytes following in vivo maturation typically involves monitoring when the female experiences a surge in luteinizing hormone (LH). Oocytes are collected after the surge is detected, such as 1, 2, 3, 4, 5, 6, 7, 8, 9, 10, 11, 12, 13, 14, 15, 16, 17, 18, 19, 20, 21, 22, 23, 24, 36, 48, 60, 72, 84, 96, 108, 120, 132, 144, 156, 168, 180 or up to 196 hours later. To obtain in vivo matured oocytes, cows are first placed under general anesthesia. The reproductive tract is exposed. The opening in the fimbriated end of the oviduct is located visually and cannulated using a flanged intramedic catheter. The catheter is held in place by a surgical ligature, which is tied using a quick-release square knot, just below the flange. The catheter is then retracted until it is stopped by the ligature. The base of the oviduct, just above the utero-tubal junction is visualized by using digital pressure to blanch the surrounding tissue. The oviductal lumen is then cannulated using a fine (23-27 gauge) hypodermic needle, which is attached to a syringe that has been pre-filled with embryo collection medium. Approximately 4 ml of collection medium is injected through the lumen of the oviduct, through the catheter and directed into a sterile plastic Petri dish. The catheter is then removed from the oviduct, the ends are rinsed into the collection dish and the catheter lumen is flushed with media using the hypodermic needle. After flushing both oviducts, the abdominal incision is closed using a 2-layer closure followed by surgical adhesive on the skin incision. Timing of the oocyte collection depends upon the stage of maturation that is desired. Ovulation time is estimated by utilizing a combination of visual observation for estrus, serum LH assays to detect the LH surge, and progesterone assays to confirm the validity of the LH surge. Alternatively, the reproductive tract of the donor may be removed and the eggs flushed from the excised oviducts. Additional means of ova extraction could be performed post mortem including aspiration of ova from follicles or flushing ovulated ova from the oviducts. Hormone administration may be useful in controlling the timing and induction of the estrus cycle and or ovulation.

While the details presented here represent common methodology for obtaining oocytes at metaphase II of meiosis, other methods may also be used to obtain mature oocytes suitable as recipients for nuclear transplantation. Oocytes may be obtained from both live and deceased animals and utilized as recipient oocytes for nuclear transplantation. Where oocytes are obtained, they may be obtained at a stage prior to or during metaphase II. It is generally envisioned that oocytes will be cultured in vitro, however, in vivo culture is also specifically contemplated. The oocytes may be cultured for 1, 2, 3, 4, 5, 6, 7, 8, 9, 10, 11, 12, 13, 14, 15,16, 17, 18, 19, 20, 21, 22, 23, 24, 36, 48, 60, 72, 84, 96, 108, 120, 132, 144, 156, 168 or 180 days or up to or more than a year. Oocytes also may be frozen; thus, such oocytes may be cultured before and/or after being frozen. Oocytes may be cultured for short or long periods of time. As mentioned above. oocytes from other species may also be used as recipient ova for nuclear transplantation. Oocytes obtained from cows have been previously used. Bovine oocytes matured to metaphase II of meiosis may be obtained using procedures similar to those described above for obtaining in vitro matured bovine oocytes. Bovine ovaries can be obtained from abattoirs and immature oocytes aspirated from follicles and matured in vitro. Alternatively they can be purchased from commercial sources.

B. Enucleation

Once an oocyte for nuclear transplantation is isolated, the nuclear material may be removed. Methods of enucleation are well known in the art, such as described in U.S. Pat. No. 4,994,384 and 5,945,577, and EP0930009A1, which are incorporated by reference herein. For example, with cows, metaphase II bovine oocytes are either placed in HECM, optionally containing 7.5 micrograms per milliliter cytochalasin B, for immediate enucleation, or may be placed in a suitable medium, for example, an embryo culture medium such as CR1aa, plus 10% estrous bovine serum. These may then be enucleated later, in some cases, not more than 24 hours after collection, and generally 16-18 hours after placing the oocytes in culture.

Prior to enucleation, the oocytes to be enucleated may be cultured. The time that the oocytes are cultured varies; they may be cultured for 6, 12, 18, 24 or more hours, or 1, 2, 3, 4, 5, 6, 7 or more days, or 1, 2, 3, 4 or more weeks after being retrieved from the donor or after being matured or in culture.

Enucleation accomplished microsurgically may use a micropipette to remove the polar body and the adjacent cytoplasm. The oocytes are then be screened to identify those of which have been successfully enucleated. Screening may be effected by staining the oocytes with 1 microgram per milliliter 33342 Hoechst dye in HECM, and then viewing the oocytes under ultraviolet irradiation for less than 10 seconds. The oocytes which are successfully enucleated can then be placed in a suitable culture medium, e.g., CR1aa plus 10% serum.

Enucleation is traditionally carried out microsurgically, nevertheless, a person of ordinary skill would be aware that successful enucleation may also be achieved, for example, chemically, by centrifugation, by autoenucleation or by irradiation. See, for example Tatham, et al. (1995), Dominko, et al. (2000), Karnikova, et al. (1998), Elsheikh, et al. (1997), Fulka, et al. (1993).

The timing of enucleation may occur immediately after retrieval or after the oocyte reaches maturation, or it may occur about 1, 2, 3, 4, 5, 6, 7, 8, 9, 10, 11, 12, 13, 14, 15, 16, 17, 18, 19, 20, 21, 22, 23, 23, or 24 or more hours after collection or thawing. Where the oocyte is cultured or cryopreserved prior to enucleation, enucleation may occur after 1, 2, 3, 4, 5, 6, 7, 8, 9, 10, 11, 12, 13, 14, 15, 16, 17, 18, 19, 20, 21, 22, 23, 24, 36, 48, 60, 72, 84, 96, 108, 120, 132, 144, 156, 168 or 180 days or up to or more than a year after collection. While oocytes in metaphase II may be used, oocytes at other stages of meiosis, such as prophase I, metaphase I, anaphase I, telophase I, prophase II, anaphase II or telophase II, are contemplated.

C. Somatic Cell Sources

It is envisioned that the somatic cells, somatic cell nuclei or somatic cell nuclear DNA being transferred to the oocytes described above may be obtained from a variety of sources within the bovine. Cell nuclei derived from embryonic, fetal, juvenile, or adult cells can be obtained from a variety of different tissue types including but not limited to skin, oral mucosa, blood, bone marrow, lung, liver, kidney, muscle, and the reproductive tract. Each different cell type selected for use may require slight variations in the methods employed for culture, expansion, freezing, thawing, handling and treatment prior to utilization for cloning. However, the examples provide a general approach that can be utilized for preparing cells obtained from skin and provides an example of the basic methodology.

Cells for somatic cell nuclear transfer may be derived from surgical or biopsy specimens. One technique is routinely used to generate cell lines from surgical or biopsy samples by harvesting cells that grow from small pieces of tissue. The other techniques are designed to minimize the growth of cells until such time as cells are needed for the nuclear transfer. These cells can be derived by causing the outgrowth of cells from tissue pieces that have been suitably cryopreserved. There are essentially three steps taken to cryopreserve cells and tissues for somatic cell nuclear transfer. These techniques have been successfully applied to the collection of cells from healthy animals and from animals that have been dead for periods ranging from hours to days. In some examples, somatic cells for nuclear transfer are isolated after 1, 2, 3, 4, 5, 6, 7, 8, 9, 10, 11, 12, 13, 14, 15, 16, 17, 18, 19, 20, or more passages. The cells are frozen, then thawed, and cultured 1, 2, 3, 4, 5, 6, 7, 8, 9, 10, or more days prior to being used as nuclear donors for nuclear transfer.

D. Nuclear Transplantation/Nuclear Transfer

Once enucleated, the oocytes are ready for implantation of alternate nuclear material by nuclear transplantation. Nuclear transfer involves the transplantation of viable nuclei or any DNA capable of being reprogrammed and directing embryonic development, from typically somatic cells to enucleated oocytes. While the nuclei are generally derived from living cells, it is specifically contemplated, as described above, that nuclei derived from non-living tissue may also be successfully transplanted. The process constitutes isolating the recipient oocytes from a donor, enucleating the oocytes, transferring the desired cell nucleus into the enucleated oocyte, e.g., by cell fusion or microinjection, to form a cybrid.

One method of fusion is electrofusion, nevertheless, fusion may alternatively be carried out by the exposure of the cells to fusion promoting chemicals, i.e. ethylene glycol, the use of an inactivated virus such as, for example, Sendai virus, or direct injection of the nucleus. The cybrid may be transferred immediately, or it may be cultured to at least the 2-cell developmental stage and then transferred into a surrogate for gestation. In the context of the instant invention, the nuclei are reprogrammed to direct the development of cloned embryos. The cloned embryos can also be combined with normal embryos, parthenogenetically-produced embryos or polyploid embryos (e.g., tetraploid embryos) to produce chimeric embryos, fetuses and/or offspring. Nuclear transfer techniques or nuclear transplantation techniques are known in the art, as set forth, for example in U.S. Pat. No. 5,945,577, Campbell et al, (1995); Collas et al, (1994); Keefer et al, (1994); Sims et al, (1993); WO 94/26884; WO 94/24274, and WO 90/03432, EP0930009A1 and U.S. Pat. Nos. 4,944,384 and 5,057,420.

In addition to the fusion of an oocyte and a nucleus/cell or nuclear DNA, the oocyte or the cybrid may be activated prior to transfer into a surrogate animal that will carry the cybrid to term. The oocyte may be activated prior to contact with the nucleus/cell or the cybrid. Activation of the cybrid may occur 10, 20, 30, 40, 50, 60, 70, 80, 90, 100, 110, 120, 130, 140, 150, 180, or more minutes after the oocyte and nucleus/cell is fused. Activation of the oocyte may occur within 1, 2, 3, 4, 5, 6, 7, 8, 9, 10 hours or 1, 2 or more days after the oocyte is retrieved (or thawed) or before or during fusion. The oocyte may be frozen before or after it is activated.

Activation may be achieved physically, electrically, or chemically. Physical activation includes pricking the oocyte or cybrid. Electrical activation involves applying an electrical pulse. Chemical activation includes the use of ionomycin. The oocyte or cybrid may be in fusion medium at the time of activation. After activation, oocytes or cybrids may be incubated in cycloheximide and/or cytochalasin B for 1, 2, 3, 4, 5, 6, 7, 8, 9, 10, or more hours between 37° C.-39° C. They may be washed after that incubation step.

Following nuclear transplantation, the embryo may be allowed to undergo divisions and develop to the morula, and then blastocyst stage, or a later fetal stage prior to implantation. In order to improve yield, the embryo may be split and the cells clonally expanded at any developmental stage. This manipulation facilitates the production of multiple embryos from a single successful nuclear transplantation. Clones from a cloned entity may be used as a source for additional cloning experiments. Embryos are generally allowed to undergo at least a first division prior to implantation by embryo transfer.

E. Embryo Transfer

Some of the techniques of embryo transfer and recipient animal management are generally standard procedures. Embryo transfer is a well known and widely used technology for the control of reproduction in agricultural animals. Traditionally females are superovulated and bred, the embryos recovered from the pregnant female and transferred to a surrogate mother. Alternatively, eggs are retrieved and mixed with sperm in a culture dish to allow fertilization. In the context of the instant invention, embryos (cybrid) are not derived from naturally fertilized ova but rather from enucleated ova which have been subjected to nuclear transplantation. The cybrid may be immediately transferred and/or implanted into a surrogate, or the transfer may occur after culturing. Where the cybrid is cultured, the cybrid may be cultured to the 2-, 4-, 8-, 16-, 32-, 64-, 128-, or 256-cell stage, or until the cybrid develops to a morula, blastocyst or later embryonic or fetal stage. Due to failure of the embryo to implant in the reproductive tract or spontaneous abortion, embryo transfers generally have a less than 50% success rate. Early embryo development normally occurs in the Fallopian tube (oviduct), however, the Fallopian tube is more than a conduit between the ovary and the uterus. The Fallopian tube supplies a complex mixture of nutrients and may help to detoxify metabolic products produced by the embryo. Thus, it is important for embryos to successfully implant to be placed in a surrogate whose Fallopian tubes or uterus are at the proper stage to receive and allow implantation of the embryo. Whether a cybrid is transferred to the Fallopian tubes or uterus depends upon the developmental stage of the cybrid. Thus, depending upon the stage of cybrid development, synchronous transfers are important for successful transplantation, i.e., the stage of the embryo is in synchrony with the estrous cycle of the recipient female. For the purpose of the invention, a synchronous surrogate, is a female who is physically capable of being implanting with a transplanted embryo and fostering embryo development. Synchrony may be achieved through selection, or a female may be induced to be in synchrony through the use of hormones or other pharmaceuticals.

F. Bovine Estrus

In order for proper oocyte isolation, and embryo implantation, the reproductive status of cows may need to be determined. The estrous cycle in the cow averages 21 days in length, but can vary between 17 and 24 days and still be considered normal. The length of the estrous cycle is measured as the time between two consecutive estrus or heat periods. The physiological and hormonal changes which occur in the female over the estrous cycle prepare the reproductive tract for estrus (the period of sexual receptivity), ovulation (release of the egg) and implantation (attachment of the fertilized egg to the uterus).

The estrus cycle may be synchronized through the administration of commercially available prostaglandin products (Lutalyse available from Upjohn Company or Estrumate, marketed by ICI Pharmaceuticals). These products function to regulate the estrus cycle by causing luteolysis.

G. NRAMP1

Natural resistance associated macrophage protein (NRAMP1), is a polytopic integral membrane protein that has structural features similar to prokaryotic and eukaryotic transporters. The 65 kD is expressed only in macrophages/monocytes and PMNs. The protein is normally localized to endosomal/lysosomal compartment and is rapidly recruited to the phagosome membrane following phagocytosis. Mutations in Nramp1 seem to abrogate the ability of macrophages to kill intracellular pathogen such as Mycobacterium, Brucella and Salmonella.

In bovines, the NRAMP1 gene is associated with the susceptibility or resistance of an animal to diseases such as brucellosis, tuberculosis, paratuberculosis and salmonellosis (See U.S. Pat. No. 6,114,118, herein expressly incorporated by reference). Bovine NRAMP1 is expressed primarily in macrophages and tissues of the reticuloendothelial system, and is predicted to encode a 548 amino acid protein that has 12 transmembrane segments with one hydrophilic N-terminal region containing a Src homology 3 (SH3)-binding motif located at the cytoplasmic surface, and a conserved consensus transport motif.

The bovine NRAMP1 gene has at least two differing sequences in the 3′ untranslated region of the gene that are significantly (P=0.0089) associated with the resistance or susceptibility of an animal to diseases, specifically intracellular pathogens, such as, for example, brucellosis, tuberculosis, paratuberculosis and salmonellosis (SEQ ID NO. 1, 2 & 3). Potential mechanisms for bovine NRAMP1 control over, or association with, resistance/susceptibility have been reviewed by others, which are incorporated herein by reference (Vidal et al. 1993; Cellier et al. 1994; Blackwell et al. 1994; Ivanyi et al. 1994; Vidal et al. 1995; Blackwell et al. 1995; Nathan 1995).

H. Bovines

The methods of the instant invention may be applied to any bovine species, for example, Bos taurus, Bos indicus, Bos gaurus, and Bos primigenius.

The Bos genus has diverged into a number of breeds through selective breeding and agricultural divergence. The instant invention is nevertheless applicable to bovines considered to belong to a recognized breed as well as bovines considered of mix-breed. A list of exemplary breeds that may be cloned or the donors of cells or cellular material for use in the context of the instant invention includes, for example: Aberdeen-Angus, Abigar, Abondance, Abyssian Highland Zebu, Abyssian Shorthorned Zebu, Aceh, Achham, Adamawa, Aden, Afghan, Africander, Africangus, Agerolese, Alambadi, Ala-Tau, Albanian, Albanian Dwarf, Alberes, Albese, Aleutian wild, Alentejana, Aliad Dinka, Alistana-Sanabresa, Alur, American Angus, American Beef Friesian, American Breed, American Brown Swiss, American White Park, Amerifax, Amritmahal, Anatolian Black, Andalusian Black, Andalusian Blond, Andalusian Grey, Angeln, Angoni, Ankina, Ankole-Watusi, Aosta, Aosta Balck Pied, Aosta Chestnut, Aosta Red Pied, Apulian Podolian, Aracena, Arado, Argentine Crillo, Argentine Friesian, Armorican, Arouquesa, Arsi, Asturian, Atpadi Mahal, Aubrac, Aulie-Ata, Aure et Saint-Girons, Australian Braford, Australian Brangus, Australian Charbray, Australain Commercial Dairy Cow, Australain Friesian Sahiwal, Australain Grey, Australian Lowline, Australian Milking Zebu, Australian Shorthorn, Australian White, Austrian Simmental, Austrian Yellow, Avetonou, Avilena, Avilena-Black Iberian, Aweil Dinka, Ayrshire, Azaouak, Azebuado, Azerbaijan Zebu, Azores, Bachaur, Baggara, Baggerbont, Bahima, Baila, Bakosi, Bakwiri, Baladi, Baltic Black Pied, Bambara, Bambawa, Bambey, Bami, Banyo, Baoule, Bapedi, Bargur, Bari, Baria (Vietnam), Baria (Madagascar), Barka, Barotse, Barra do Cuanzo, Barrosa, Barroso, Barzona, Bashi, Basuto, Batanes Black, Batangas, Batawana, Bavenda, Bazadais, Bearnais, Beefalo, Beefmaker (US), Beefmaker (Aussie), Beefmaster, Beef Shorthorn, Beef Synthetic, Beijing Black Pied, Beiroa, Beja, Belgian Black Pied, Belgian Blue, Belgian Red, Belgian Red Pied, Belgian White-and-Red, Belmont Red, Belted Galloway, Belted Welsh, Bengali, Bericiana, Berrendas, Bestuzhev, Betizuak, Bhagnari, Biamal, Black Baldy, Black Forest, Black Iberian, Blanco Orejinergo, Blauw and Blauwbont, Bleu du Nord, Blonde d'Aquitaine, Blonde du Sud-Ouest, Bolivian Criollo, Bonsmara, Boran, Borgou, Boreno Zebu, Braford, Bragado do Sorraia, Braganca, Brahman, Brahmin, Brahom, Bralers, Bra-Maine, Brahmousin, Brandrood Ijsselvee, Brangus, Bra-Swiss, Bravon, Brazilian Dairy Hybrid, Brazilian Gir, Brazilian Polled, Brazilian Zebu, Breton Black Pied, British Dane, British Friesian, British Holstein, British Polled Hereford, British White, Brown Atlas, Brownsind, Bulgarian Brown, Bulgarian Red, Bulgarian Simmental, Burlina, Burmese, Burwash, Busa, Bushuev, Butana, Byelorussian Red, Byelorussian Synthetic, Cabannina, Cachena, Caiua, Calabrian, Cadeano, Caldelana, Calvana, Camargue, Cambodian, Canadien, Canary Island, Canchim, Cape Bon Blond, Caracu, Carazebu, Cardena, Carpathian Brown, Carrena, Casanareno, Cash, Casina, Castille-Leon, Caucasian, Caucasian Brown, Central American Dairy Criollo, Central Asian Zebu, Central Russian Black Pied, Chagga, Chan-Doc, Chaouia, Cahqueno, Charbray, Charford, Charolais, Charollandrais, Char-Swiss, Charwiss, Cheju, Chernigov, Chesi, Cheurfa, Chiangus, Chianina, Chiford, Chimaine, Chinampo, Chinese Black-and-White, Chino Santandereano, Chittagong, Cholistani, Cildir, Cinisara, Colombian Criollo, Coopelso 93, Cornigliese, Corriente, Corsican, Costeno con Cuernos, Cretan Lowland, Cretan Mountain, Croatian Red, Cuban Criollo, Cuban Zebu, Cukurova, Cuprem Hybrid, Curraleiro, Cutchi, Cyprus, Czech Pied, Dabieshan, Dacca-Faridpur, Dagestan Mountains, Dairy Gir, Dairy Shorthorn, Dairy Synthetic, Dairy Zebu of Uberaba, Dajjal, Damara, Damascus, Damietta, Danakil, Dangi, Danish Red Pied, Danish Blue-and-White, Danish Jersey, Danish Red, Danish Red Pied, Dashtiara, Dengchuan, Deoni, Devarakota, Devni, Devon, Dexter, Dexter-Kerry, Dhanni, Diali, Didinga, Dishti, Djakore, Dneiper, Doayo, Dobrogea, Dongola, Doran, Dorna, Dortyol, Drakensberger, Droughtmaster, Dun Galloway, Dutch Belted, Dutch Black Pied, East African Zebu, East Anatolian Red, East Anatolian Red and White, Eastern Nuer, East Finnish, East Friesian, East Macedonian, Ecuador Criollo, Egyptian, Enderby Island Shorthorn, Epirus, Estonian Black Pied, Estonian Native, Estonian Red, Ethiopian Boran, Faeroes, Fellata, Ferrandais, Fighting Bull, Finnish, Finnish Ayrshire, Flemish, Flemish Red, Florida Scrub, Fogera, Fort Cross, Franqueiro, Frati, French Brown, French Friesian, Friesland, Frijolillo, FRS, Gacko, Gado da Terra, Galician Blond, Galloway, Gambian N'Dama, Gaolao, Garfagnina, Garre, Gasara, Gascon, Gelbvieh, Georgian Mountain, German Angus, German Black Pied, German Black Pied Dairy, German Brown, German Red, German Red Peid, German Shorthorn, German Simmental, Ghana Sanga, Ghana Shorthorn, Gir, Giriama, Girolando, Glan, Glan-Donnersberg, Gloucester, Gobra, Gole, Golpayegani, Goomsur, Gorbatov Red, Goryn, Grati, Greater Caucasus, Greek Shorthorn, Greek Steppe, Grey Alpine, Greyman, Groningen Whitehead, Grossetana, Guadiana Spotted, Gaunling, Guelma, Guernsey, Gujamavu, Guzera, Guzerando, Hainan, Halhin, Hallikar, Hariana, Harton, Harz, Hatton, Hawaiian wild, Hays Converter, Hereford, Hereland, Herens, Highland, Hinterland, Hissar, Holgus, Holmonger, Holstein, Horro, Hrbinecky, Huangpi, Huertana, Humbi, Hungarian Grey, Hungarian Pied, Hungarfries, Ibage, Icelandic, Illawarra, Ilocos, Iloilo, Improved Rodopi, Indo-Brazilian Zebu, Ingessana, Inkuku, INRA 9, Iraqi, Irish Moiled, Iskar, Israeli Friesian, Istoben, Istrian, Italian Brown, Italian Friesian, Italian Red Pied, Jamaica Black, Jamica Brahman, Jamica Hope, Jamica Red, Japanese Black, Japanese Brown, Japanese Native, Japanese Poll, Japanese Shorthorn, Jarmelista, Jaulan, Javanese, Javanese Ongole, Javanese Zebu, Jellicut, Jem-Jem Zebu, Jenubi, Jerdi, Jersey, Jersian, Jersind, Jiddu, Jijjiga Zebu, Jinnan, Jochberg, Jotko, Kabota, Kabyle, Kachcha Siri, Kalakheri, Kalmyk, Kamasia, Kamba, Kamdhino, Kandahari, Kanem, Kangayam, Kaningan, Kankrej, Kaokoveld, Kappiliyan, Kapsiki, Karamajong, Karan Fries, Karan Swiss, Katerini, Kavirondo, Kazkh, Kazkh Whitehead, Kedah-Kelantan, Kenana, Kenkatha, Kenran, Kenya Boran, Kenya Zebu, Kerry, Keteku, Khamala, Kherigarh, Khevsurian, Khillari, Kholmogory, Khurasani, Kigezi, Kikuyu, Kilara, Kilis, Kinniya, Kisantu, Kochi, Kolubara, Konari, Korean Native, Kostroma, Kravarsky, Krishnagiri, Krishina Valley, Kuchinoshima, Kumamoto, Kumauni, Kurdi, Kurgan, Kuri, Kyoga, Ladakhi, Lagune, Lakenvelder, Las Bela, Latuka, Latvian Blue, Latvian Brown, La Velasquez, Lavinia, Lebanese, Lebedin, Lesser Caucasus, Liberian Dwarf, Libyan, Lim, Limiana, Limousin, Limpurger, Lincoln Red, Lithuanian Red, Llanero, Lobi, Local Indian Dairy, Lohani, Longhorn, Lourdais, Lowline, Lucanian, Lucerna, Lugware, Luing, Luxi, Macedonian Blue, Madagascar Zebu, Madaripur, Madura, Magal, Maine-Anjou, Makaweli, Malawi Zebu, Malnad Gidda, Malselv, Maltese cow, Malvi, Mampati, Manapari, Mandalong Special, Mangwato, Mantiqueira, Marchigiana, Maremmana, Marianas, Marinhoa, Maronesa, Maryuiti, Masai, Mashona, Matabele, Maure, Mauritius Creole, Mazandarani, Mazury, Meknes Black Pied, Menufi, Merauke, Mere, Mertolenga, Messaoria, Metohija Red, Meuse-Rhine-Yssel, Mewati, Mezzalina, Mhaswald, Milking Devon, Milking Shorthorn, Mingrelian Red, Minhota, Miniature Hereford, Miniature Zebu, Minocran, Mirandesa, Mishima, Modenese, Modicana, Moi, Monchina, Mongalla, Mongolian, Montafon, Montbeliard, Morang, Morenas del Noroeste, Morucha, Mottled Hill, Mozambique Angoni, Mpwapwa, Munshigunj, Murcian, Murgese, Murle, Murnau-Werdenfels, Murray Grey, Muris, Muturu, Nagori, Nakali, Nama, Nandi, Nantais, Nanyang, Ndagu, N'Dama, N'Dama Sanga, Nejdi, Nelore, Nepalese Hill, N'Gabou, Nganda, N'Gaoundere, Nguni, Nilotic, Nimari, Nkedi, Nkone, Norrnande, Normanzu, North Bangladesh, North Finnish, North Malawi Zebu, North Somali, Norwegian Red, Nuba Mountain, Nuer, Nuras, Nyoro, Okayama, Ongole, Oran, Orapa, Oulmes Blond, Ovambo, Pabna, Pajuna, Palmera, Pakota Red, Pantaneiro, Pantelleria, Paphos, Parthenias, Pechora, Pee Wee, Peloponnesus, Perijanero, Pester, Philippine Native, Piedmont, Pie Rouge de l'Est, Pie Rouge des Plaines, Pinzgauer, Pinzhou, Pisana, Pitangueiras, Polish Black-and-White Lowland, Polish Red-and-White Lowland, Polish Simmental, Polled Charolais, Polled Gir, Polled Guzera, Polled Hereford, Polled Jersey, Polled Lincoln Red, Polled Nelore, Polled Shorthorn (US), Polled Simmental, Polled Sussex, Polled Welsh Black, Polled Zebu, Poll Friesian, Poll Hereford, Poll Shorthorn (Aussie), Pontremolese, Ponwar, Porto Amboim, Posavina, Preti, Prewakwa, Puerto Rican, Pul-Mbor, Punganur, Purnea, Pyrenean, Qinchuan, Quasah, Ramgarhi, Ramo Grande, Rana, Randall Lineback, Ranger, Rath, Raya-Azebo, Red and White Friesian, Red and White Holstein, Red Angus, Red Belted Galloway, Red Bororo, Red Brangus, Red Chianina, Red Desert, Red Galloway, Red Kandhari, Red Poll, Red Sindhi, Red Steppe, Reggiana, Regus, Rendena, Renitelo, Retinta, Rhaetian Grey, Rio Limon Dairy Criollo, Riopardense, Rodopi, Rojhan, Romagnola, Roman, Romana Red, Romanian Brown, Romanian Red, Romanian Simmental, Romanian Steppe, Romosinuano, Russian Black Pied, Russian Brown, Russian Simmental, Rustaqi, RX3, Sabre, Sahford, Sahiwal, Saidi, Salers, Salorn, Sanhe, San Martinero, Santa Gertrudis, Sarabi, Sardinian, Sardinian Brown, Sardo-Modicana, Savinja Grey, Sayaguesa, Schwyz-Zeboid, Seferihisar, Senepol, Sengologa, Serbo-Cro Pied, Serbo-Cro Pinzau, Serere, Seshaga, Shahabadi, Shakhansurri, Shandong, Sharabi, Sheko, Shendi, Shetland, Shimane, Shkodra, Shuwa, Siberian Black Pied, Siberian White, Siboney, Simbrah, Simford (Australia), Simford (Israel), Simmalo, Simmental, Sinhala, Sirn, Sistani, Slovakian Pied, Slovakian Pinzgau, Slovenian Brown, Slovenian Podolian, Small East African Zebu, Socotra, Sokoto Gudali, Somali, Somba, Sonkheri, Son Valley, South African Brown Swiss, South Anatolian Red, South China Zebu, South Devon, Southern Tswana, Southern Ukrainian, South Malawi Zebu, Spanish Brown, Spreca, Sudanese Fulani, Suia, Suisbu, Suk, Suksun, Sunkuma, Sunandini, Sussex, Swedish Ayrshire, Swedish Friesian, Swedish Jersey, Swedish Mountain, Swedish Polled, Swedish Red-and-White, Swiss Black Pied, Swiss Brown, Sychevka, Sykia, Tabapua, Tagil, Taino, Taiwan Zebu, Tajma, Tamankaduwa, Tambov Red, Tanzanian Zebu, Tarai, Tarentaise, Tarina, Taylor, Telemark, Texas Longhorn, Thai, Thailand Fighting cow, Thanh-Hoa, Thari, Thatparkar, Thessaly, Thibar, Thillari, Tibetan, Tinima, Tinos, Tonga, Toposa, Toro, Toronke, Tottori, Toubou, Toupouri, Transylvanian Pinzgua, Tropical, Tropical Dairy Cattle, Tropicana, TSSHZ-1, Tawana, Tudanca, Tuli, Tuni, Turino, Tukana, Turkish Brown, Turkish Grey Steppe, Turkmen, Tux-Zillertal, Tuy-Hoa, Tyrol Grey, Uganda Zebu, Ujumqin, Ukrainian Grey, Ukrainian Whiteheaded, Umblachery, Ural Black Pied, Valdres, Vale and Vaalbonte, Vaynol, Vendee Marsh, Venezuela Criollo, Venezuelan Zebu, Verinesa, Vianesa, Victoria, Vietnamese, Villard-de-Lans, Vogelsberg, Volnsk, Voderwald, Vosges, Wakwa, Watusi (USA), Welsh Black, Wenshan, West African Dwarf Shorthorn, West African Shorthorn, West Finnish, West Macedonian, Whitebred Shorthorn, White Caceres, White Fulani, White Galloway, White Nile, White Park, White Sange, White Welsh, Witrik, Wodabe, Wokalup, Xinjiang, Xuwen, Yacumeno, Yakut, Yanbian, Yaroslavl, Yellow Franconian, Yemeni Zebu, Yunnan Zebu, Yurino, Zambia Angoni, Zanzibar Zebu, Zaobei, Zavot, Znamensk.

I. TAES/TAMU #86 BULL

The genotypic and phenotypic characteristics of TAES/TAMU #86 BULL and polymorphisms within the NRAMP1 gene have been comprehensively reviewed (U.S. Pat. No. 6,114,118; Harmon, 1985; Harmon, 1987; Breider, 1987; Templeton, 1988; Harmon, 1988; Harmon, 1989; Price, 1990; Smith, 1990; Smith, 1990; Estes, 1990; Smith, 1990; Estes, 1990; Davis, 1990; Adams, 1990; Templeton, 1990; Price, 1990; Smith, 1990; Campbell, 1992; Price, 1993; Campbell, 1994; Qureshi, 1996; Feng, 1996; Adams, 1998; Horin, 1999; Adams, 1999; Barthel, 2000, all herein expressly incorporated by reference). Briefly, TAES/TAMU #86 was a black angus bull that died in 1997. During the course of research relating to natural resistance to infection, this animal was determined to be remarkably resistant to infection with intracellular pathogens, specifically mediators of bovine brucellosis, tuberculosis, paratuberculosis and salmonellosis. This resistance was linked, at least in part to a polymorphism within the animals 3′ UTR of the NRAMP1 gene. It is hypothesized that this polymorphism may effect expression levels of the mature protein.

The polymorphism results from a transversion at position 1782 of the bovine NRAMP1 cDNA; thymine in the resistant sequence to guanine in the susceptible sequence. Additionally, there is a polymorphic DNA microsatellite sequence difference between resistant and susceptible cattle involving the number of (GT) dinucleotide repeats and spacing in the 3′ UTR of bovine NRAMP 1 beginning at position 1779 of the cDNA.

J. Cryopreservation

Cryopreservation methods include rapid vitrification (30,000° C./minute), slow vitrification (8,000° C./minute), rapid freezing (100° C./minute), and slow freezing (0.5° C./minute). The method of cryopreservation used is important due to the freezing of intracellular water. During slow cooling, water leaves the cell because of the osmotic imbalance caused by the lower concentration of water in the extracellular environment due to ice formation. The increase in solute concentration due to the decrease in water volume can be harmful. Alternatively, too much water in the inside the cell can lead to damage during warming. Cryoprotectants protect cells by diluting salt that becomes more and more concentrated as ice forms. They also stabilize membranes and proteins and reduce the intracellular ice formation temperatures. Cell survival is low at very slow and very fast cooling rates (U.S. Pat. No. 5,891,617).

Dimethylsulfoxide (DMSO) and glycerol are the most commonly used cryoprotectants. DMSO causes a depression in the freezing point and therefore increased water removal from the cell prior to freezing. DMSO generates heat when dissolved in aqueous solutions. It must be diluted and allowed to cool before addition to cells. DMSO and glycerol are usually used in a 5-10% solution in growth medium. They are not used together except for cryopreserving plant cells. Cells may be incubated in the cryoprotectant before beginning the cooling process. This is called the equilibration period. Cooling rates of 1° C. per minute and the use of a cryoprotective agent are commonly used to protect the cells. Larger cells generally require greater control of cooling rates. Frozen cells should be maintained below −130° C.

When thawing of the sample is desired, warming should occur as quickly as possible. This is generally achieved by placing the vial into 37° C. water. The outside of the vial is disinfected before the vial is opened to protect against contamination of the sample. The cells are then transferred to fresh growth medium to decrease the concentration of the cryoprotectant. The cells can be centrifuged and the supernatant removed. The cells are then resuspended in fresh growth medium (Simione, F. P. (1998)).

PCT Patent Application No. PCT/US92/00599 describes a tissue preservation method in which dissected heart valve, veins and musculoskeletal connective tissue are divided into at least two portions that are contaminated with microbes to various degrees. The group containing a lower level of contamination is exposed to an antimicrobial regimen and cryopreserved. The group containing the higher level of contamination is exposed to a second antimicrobial regimen and cryopreserved. This reference is directed to the decrease of microbial contamination of heart valves, veins and musculoskeletal connective tissue. The antimicrobial regimen lasts preferably 4 hours.

U.S. Pat. No. 5,891,617 describes cryopreservation of harvested mammalian tissues and cultured tissue equivalents in a cryoprotectant solution. Solutions contain a cell penetrating glass forming agents such as propylene glycol, ethylene glycol, and dimethylsulfoxide or non-cell penetrating glass forming agents such as high molecular weight complex carbohydrates. These solutions are diluted in a base at physiological pH. The preferred solution is 2 M glycerol in Dulbecco's Modified Eagle's Medium (DMEM).

U.S. Pat. No. 5,964,096 describes a method and package design for cryopreservation and storage of cultured tissue equivalents. The cryopreservation method includes immersing the tissue in cryoprotectant solution, agitating the sample, cooling to solid-liquid phase equilibrium temperature for the cryoprotectant, seeding extracellular ice, and freezing the tissue to a temperature below −70° C. Cryopreserved tissue is warmed at a high rate by direct application of a warmed media or buffered solution or other heating method. Cryoprotectant is removed form the thawed tissue before use. Also described is a package that has an improved heat transfer rate, allowing for a more controlled cooling and heating process.

l. Retrieval Of Tissues Or Cells From Cryopreservation

Tissue pieces or cells can be recovered from cryopreservation and retain high viability if the tissue is maintained at liquid nitrogen temperatures continuously. It is important not to hold ampule out of nitrogen for more than a few seconds at a time when searching for ampule of cells in the cryobank.

When retrieving cells or tissue pieces, have laminar flow hood decontaminated and all media warmed and culture dishes ready.

Remove ampule from liquid nitrogen and drop directly into a 37° C. water filled beaker (˜500ml) to provide a large heat sink. Cells should be rapidly thawed by gently swirrelling ampule with fingers around the water to facilitate rapid warming.

Once thawed, decontaminate outside of cryotube only after recording all data listed on the cryotube. Solvents can remove information on label.

Gently resuspend frozen cells in at least 10 ml of fresh medium without DMSO and place medium with cells/tissue in a 25 cm² flask with 7-10 ml of media or with 20 ml in 75 cm² tissue culture flask. Thawed tissues can be transferred to 10 ml of fresh medium without DMSO in a conical centrifuge tube to eliminate DMSO. Tissue pieces can be collected by velocity sedimentation at unit gravity or gentle centrifugation then resuspended in fresh medium before transfer to the flask. Return flask to incubator at 37° C. under a 5% CO₂ atmosphere with cap loose.

Depending on the rate of plating, replace media containing residual DMSO with fresh medium as soon as possible, but at the latest, within 8 hr.

Record all information on flask including passage number, transfer no., the number of times the tissue piece has been frozen and thawed, etc.

K. Nucleic Acids

In the context of the instant invention, it may be important to screen developing embryos and postnatal animals for genetic identity to the donor organism. In the context of transgenic animals, it the following techniques will be useful in screening for the presence of the transgene. This screening process may require the isolation, amplification and/or manipulation of genetic material. The following sections provide a brief overview of nucleic acids, their function and manipulation.

In the context of the instant invention, genes are sequences of DNA in an organism's genome encoding information that is converted into various products making up a whole cell. They are expressed by the process of transcription, which involves copying the sequence of DNA into RNA. Most genes encode information to make proteins, but some encode RNAs involved in other processes. If a gene encodes a protein, its transcription product is called mRNA (“messenger” RNA). After transcription in the nucleus (where DNA is located), the mRNA must be transported into the cytoplasm for the process of translation, which converts the code of the mRNA into a sequence of amino acids to form protein. In order to direct transport into the cytoplasm, the 3′ ends of mRNA molecules are post-transcriptionally modified by addition of several adenylate residues to form the “polyA” tail. This characteristic modification distinguishes gene expression products destined to make protein from other molecules in the cell, and thereby provides one means for detecting and monitoring the gene expression activities of a cell.

The term “nucleic acid” will generally refer to at least one molecule or strand of DNA, RNA or a derivative or mimic thereof, comprising at least one nucleobase, such as, for example, a naturally occurring purine or pyrimidine base found in DNA (e.g. adenine “A,” guanine “G,” thymine “T” and cytosine “C”) or RNA (e.g. A, G, uracil “U” and C). The term “nucleic acid” encompass the terms “oligonucleotide” and “polynucleotide.” The term “oligonucleotide” refers to at least one molecule of between about 3 and about 100 nucleobases in length. The term “polynucleotide” refers to at least one molecule of greater than about 100 nucleobases in length. These definitions generally refer to at least one single-stranded molecule, but in specific embodiments will also encompass at least one additional strand that is partially, substantially or fully complementary to the at least one single-stranded molecule. Thus, a nucleic acid may encompass at least one double-stranded molecule or at least one triple-stranded molecule that comprises one or more complementary strand(s) or “complement(s)” of a particular sequence comprising a strand of the molecule. As used herein, a single stranded nucleic acid may be denoted by the prefix “ss”, a double stranded nucleic acid by the prefix “ds”, and a triple stranded nucleic acid by the prefix “ts.”

Nucleic acid(s) that are “complementary” or “complement(s)” are those that are capable of base-pairing according to the standard Watson-Crick, Hoogsteen or reverse Hoogsteen binding complementarity rules. As used herein, the term “complementary” or “complement(s)” also refers to nucleic acid(s) that are substantially complementary, as may be assessed by the same nucleotide comparison set forth above. The term “substantially complementary” refers to a nucleic acid comprising at least one sequence of consecutive nucleobases, or semiconsecutive nucleobases if one or more nucleobase moieties are not present in the molecule, are capable of hybridizing to at least one nucleic acid strand or duplex even if less than all nucleobases do not base pair with a counterpart nucleobase. In certain embodiments, a “substantially complementary” nucleic acid contains at least one sequence in which about 70%, about 71%, about 72%, about 73%, about 74%, about 75%, about 76%, about 77%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, to about 100%, and any range therein, of the nucleobase sequence is capable of base-pairing with at least one single or double stranded nucleic acid molecule during hybridization. In certain embodiments, the term “substantially complementary” refers to at least one nucleic acid that may hybridize to at least one nucleic acid strand or duplex in stringent conditions. In certain embodiments, a “partly complementary” nucleic acid comprises at least one sequence that may hybridize in low stringency conditions to at least one single or double stranded nucleic acid, or contains at least one sequence in which less than about 70% of the nucleobase sequence is capable of base-pairing with at least one single or double stranded nucleic acid molecule during hybridization.

Screening methods of cells and/or organisms for the determination of effective nuclear transplantation may be carried out by hybridization screening, PCR, RFLP, Northern Blotting, Southern Blotting or other methods that a person of ordinary skill would view as adequate to determine nucleic acid identity. A brief description of exemplary methods follows.

1. Hybridization

Hybridization is understood to mean the forming of a double stranded molecule and/or a molecule with partial double stranded nature. Stringent conditions are those that allow hybridization between two homologous nucleic acid sequences, but precludes hybridization of random sequences. For example, hybridization at low temperature and/or high ionic strength is termed low stringency. Hybridization at high temperature and/or low ionic strength is termed high stringency. Low stringency is generally performed at 0.15 M to 0.9 M NaCl at a temperature range of 20° C. to 50° C. High stringency is generally performed at 0.02 M to 0.15 M NaCl at a temperature range of 50° C. to 70° C. It is understood that the temperature and/or ionic strength of a desired stringency are determined in part by the length of the particular probe, the length and/or base content of the target sequences, and/or to the presence of formamide, tetramethylammonium chloride and/or other solvents in the hybridization mixture. It is also understood that these ranges are mentioned by way of example only, and/or that the desired stringency for a particular hybridization reaction is often determined empirically by comparison to positive and/or negative controls.

Accordingly, the nucleotide sequences of the disclosure may be used for their ability to selectively form duplex molecules with complementary stretches of genes and/or RNA. Depending on the application envisioned, it is preferred to employ varying conditions of hybridization to achieve varying degrees of selectivity of probe towards target sequence.

Nucleic acid molecules having sequence regions consisting of contiguous nucleotide stretches of about 13, 14, 15, 16, 17, 18, 20, 25, 30, 35, 40, 45, 50, 55, 60, 70, 80, 90, 100, 125, 150, 175, 200, 250, 300, 350, 400 or more basepairs (bp) to about 5000 bp, or even up to and including sequences of about 30-50 cM or so, identical or complementary to the target DNA sequence, are particularly contemplated as hybridization probes for use in embodiments of the instant invention. It is contemplated that long contiguous sequence regions may be utilized including those sequences comprising about 100, 200, 300, 400, 500, 600, 700, 800, 900, 1000, 1100, 1200, 1300, 1400, 1500, 2000, 2500, 3000, 3500, 4000, 4500, 5000, 5500, 6000, 6500, 7000, 7500, 8000, 8500, 9000, 9500, 10,000 or more contiguous nucleotides or up to and including 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more cM.

As used herein “stringent condition(s)” or “high stringency” are those that allow hybridization between or within one or more nucleic acid strand(s) containing complementary sequence(s), but precludes hybridization of random sequences. Stringent conditions tolerate little, if any, mismatch between a nucleic acid and a target strand. Such conditions are well known to those of ordinary skill in the art, and are preferred for applications requiring high selectivity. Non-limiting applications include isolating at least one nucleic acid, such as a gene or nucleic acid segment thereof, or detecting at least one specific mRNA transcript or nucleic acid segment thereof, and the like.

For applications requiring high selectivity, it is preferred to employ relatively stringent conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe and/or the template and/or target strand, and/or would be particularly suitable for isolating specific genes and/or detecting specific mRNA transcripts. It is generally appreciated that conditions may be rendered more stringent by the addition of increasing amounts of formamide.

2. Polymerase Chain Reaction

The technique of“polymerase chain reaction,” or “PCR,” as used herein generally refers to a procedure wherein minute amounts of a specific piece of nucleic acid, RNA and/or DNA, are amplified as described in U.S. Pat. Nos. 4,683,195; 4,683,202; and 4,683,194, which are herein expressly incorporated by reference. Generally, sequence information from the ends of the region of interest or beyond needs to be available, such that oligonucleotide primers can be designed; these primers will be identical or similar in sequence to opposite strands of the template to be amplified. The 5′ terminal nucleotides of the two primers may coincide with the ends of the amplified material. PCR can be used to amplify specific RNA sequences, specific DNA sequences from total genomic DNA, and cDNA transcribed from total cellular RNA, bacteriophage or plasmid sequences, etc. See generally Mullis et al., (1989). As used herein, PCR is considered to be one, but not the only, example of a nucleic acid polymerase reaction method for amplifying a nucleic acid test sample, comprising the use of a known nucleic acid (DNA or RNA) as a primer and utilizes a nucleic acid polymerase to amplify or generate a specific piece of nucleic acid or to amplify or generate a specific piece of nucleic acid that is complementary to a particular nucleic acid.

3. Northern and Southern Blotting

Blotting techniques are well known to those of skill in the art. Southern blotting involves the use of DNA as a target, whereas Northern blotting involves the use of RNA as a target. Each provide different types of information, although cDNA blotting is analogous, in many aspects, to blotting or RNA species.

Briefly, a probe is used to target a DNA or RNA species that has been immobilized on a suitable matrix, often a filter of nitrocellulose. The different species should be spatially separated to facilitate analysis. This often is accomplished by gel electrophoresis of nucleic acid species followed by “blotting” on to the filter.

Subsequently, the blotted target is incubated with a probe (usually labeled) under conditions that promote denaturation and rehybridization. Because the probe is designed to base pair with the target, the probe will binding a portion of the target sequence under renaturing conditions. Unbound probe is then removed, and detection is accomplished as described above.

4. Restriction Fragment Length Polymorphism

“Restriction Enzyme Digestion” of DNA refers to catalytic cleavage of the DNA with an enzyme that acts only at certain locations in the DNA. Such enzymes are called restriction endonucleases, and the sites for which each is specific is called a restriction site. The various restriction enzymes used herein are commercially available and their reaction conditions, cofactors, and other requirements as established by the enzyme suppliers are used. Restriction enzymes commonly are designated by abbreviations composed of a capital letter followed by other letters representing the microorganism from which each restriction enzyme originally was obtained and then a number designating the particular enzyme. In general, about 1 μg of plasmid or DNA fragment is used with about 1-2 units of enzyme in about 20 μl of buffer solution. Appropriate buffers and substrate amounts for particular restriction enzymes are specified by the manufacturer. Incubation of about 1 hour at 37° C. is ordinarily used, but may vary in accordance with the supplier's instructions.

Restriction fragment length polymorphisms (RFLPs) analysis capitalizes on the selectivity of restriction enzymes to detect the genetic changes in specific loci. RFLP are genetic differences detectable by DNA fragment lengths, typically revealed by agarose gel electrophoresis, after restriction endonuclease digestion of DNA. There are large numbers of restriction endonucleases available, characterized by their nucleotide cleavage sites and their source, e.g., Eco RI. Variations in RFLPs result from nucleotide base pair differences which alter the cleavage sites of the restriction endonucleases, yielding different sized fragments. Means for performing RFLP analyses are well known in the art.

As described in U.S. Pat. No. 5,580,729, herein expressly incorporated by reference, one means of testing for loss of an allele is by digesting the first and second DNA samples of the neoplastic and non-neoplastic tissues, respectively, with a restriction endonuclease. Restriction endonucleases are well known in the art. Because they cleave DNA at specific sequences, they can be used to form a discrete set of DNA fragments from each DNA sample. The restriction fragments of each DNA sample can be separated by any means known in the art. For example, an electrophoretic gel matrix can be employed, such as agarose or polyacrylamide, to electrophoretically separate fragments according to physical properties such as size. The restriction fragments can be hybridized to nucleic acid probes which detect restriction fragment length polymorphisms, as described above. Upon hybridization hybrid duplexes are formed which comprise at least a single strand of probe and a single strand of the corresponding restriction fragment. Various hybridization techniques are known in the art, including both liquid and solid phase techniques. One particularly useful method employs transferring the separated fragments from an electrophoretic gel matrix to a solid support such as nylon or filter paper so that the fragments retain the relative orientation which they had on the electrophoretic gel matrix. The hybrid duplexes can be detected by any means known in the art, for example, the hybrid duplexes can be detected by autoradiography if the nucleic acid probes have been radioactively labeled. Other labeling and detection means are known in the art and may be used in the practice of the present invention.

Nucleic acid probes which detect restriction fragment length polymorphisms for most non-acrocentric chromosome arms are available from the American Type Culture Collection, Rockville, Md. These are described in the NIH Repository of Human DNA Probes and Libraries, published in August, 1988. Methods of obtaining other probes which detect restriction fragment length polymorphisms are known in the art. The statistical information provided by using the complete set of probes which hybridizes to each of the non-acrocentric arms of the human genome is useful prognostically. Other subsets of this complete set can be used which also will provide useful prognostic information. Other subsets can be tested to see if their use leads to measures of the extent of genetic change which correlates with prognosis, as does the use of the complete set of alleles.

5. Mitochondrial DNA

In order to insure that effective nuclear transplantation has occurred, it may be necessary to screen both nuclear and cytoplasmic (mitochondrial) DNA. Mitochondria are the source of cytoplasmic genetic information. Mitochondria carry multiple copies of a circular genome that is replicated and expressed within the organelle and is inherited maternally. It is the only genetic element known to be inherited cytoplasmically from the maternal oocyte in mammals. Mitochondrial DNA in animals codes for 13 polypeptides that have been identified as components of the ATP synthesis system and codes for all of the transfer RNAs used in mitochondrial protein synthesis. The only non-coding portion of the genome is a region of approximately 900 base pairs that is referred to as the displacement loop or “D-loop”. This D-loop is involved in the control of transcription and replication of mitochondrial DNA. Variation in mitochondrial DNA will affect the size of the mitochondrial DNA population in the cell, abundance of mitochondrial DNA gene transcripts and translation products, and mitochondrial oxidative energy transduction capacity.

A variety of methods exist for isolating mitochondrial DNA, such as, for example, U.S. Pat. No. 5,292,639, Lindberg, et al. 1992 and Koehler et al., 1988, and incorporated herein by reference. Briefly, to isolate mitochondrial DNA from blood, leukocytes are isolated from anticoagulated blood by low-speed centrifugation after erythrocyte lysis with 140 mM ammonium chloride, pH 7.4. Leukocytes are lysed with 1% wt/vol Triton X-100 in the presence of 1% wt/vol sodium dodecyl sulfate. Nuclei and cell membranes are separated by the cytosolic fraction by centrifugation at 12,000×g for 5 min. Soluble proteins are extracted from the resulting supernatant with 1:1 phenol:chloroform, and nucleic acids precipitated from the aqueous phase with 3 vol. ethanol. The procedure yields supercoiled and relaxed covalently closed and nicked circular mitochondrial DNA molecules, RNA and only trace amounts of nuclear DNA contaminants (U.S. Pat. No. 5,292,639).

Mitochondrial DNA may be manipulated and analyzed with the methods described in the previous sections as well as other techniques known to one of ordinary skill.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

Transplantation of Cybrids into Surrogate Cows for the Production of Cloned Bovine Embryos

A. Material and Methods

Acquisition of Unfertilized Oocytes for Nuclear Transplantation

In order to obtain ovulated oocytes for use as recipient ova for nuclear transfer, immature ova were collected from the slaughterhouse and matured for 20 hours in TCM 199 (Gibco) supplemented with 10% fetal calf serum (FCS; Gibco), 0.1 units/ml FSH (Sioux Biochem, Sioux City, Iowa), 0.1 units/ml LH (Sioux Biochem), 1 μg/ml estradiol (Sigma, St Louis, Mo.), 28 μg/ml pyruvate (Sigma), 0.05 μg/ml EGF (Sigma) and 1% Pen/Strep, in an atmosphere of 5% CO₂ and air at 39° C. The cumulus-oocyte complexes were removed by vortexing 20 hours post maturation (hpm) for 3 minutes in 0.1% hyaluronidase TL-Hepes then the oocytes were washed and placed in TCM 199+10% FCS.

Acquisition of Nucleus Donor Cells, Cell Culture and Preparation of Donor Cells

In 1985, adult fibroblast cells were isolated from ear punch obtained from Texas A&M bull no. 86 (TAES/TAMU #86) cultured in DMEM supplemented with 10% FBS, then frozen in LN₂ for storage. These fibroblasts, which were about 15 years old, were thawed and placed into cultured prior to nuclear transfer by serum starvation in DMEM/F12+0.5% FBS for up to 5 days prior to NT. Prior to recombination, the cultured donor cells were trypsinized with trypsin-EDTA solution (Sigma, T-4174 X10) for less than 1 minute and detached by gentle pipetting. Cells were washed with 3 ml of hepes buffered TCM 199 supplemented with 10% FCS then transferred to a 15 ml centrifuge tube, (X200 g 3min) and resuspended in the appropriate buffer.

Production of Cloned Embryos by Nuclear Transplantation

For nuclear transplantation, unfertilized oocytes were obtained as described above. Oocytes were enucleated at 21 hours post in vitro maturation. Where necessary, the oocytes were denuded (cumulus cells removed) by expiring them up and down in a mouth pipette in 0.5% hyaluronidase solution (Sigma, St Louis, Mo.) for 5 minutes, and washed three times in 20 mM Hepes buffered TCM199 (GibcoBRL, Grand Island, N.Y.) supplemented with 10% FBS (HyClone, Logan Utah) (operating medium). Prior to enucleation, oocytes were placed for 10 minutes in hepes buffered TCM 199 supplemented with 10 % FCS, 5.0 μg/ml cytochalasin B (Sigma) and 5 μg/ml Hoechst 33342 (Sigma). All oocytes were carefully selected for presence of a polar body and homogeneous cytoplasm. Oocytes were transferred into drops of operating medium contained within a Petri dish and covered with mineral oil (Sigma). Enucleation was performed under a compound microscope by removing the first polar body and a small part of oocyte cytoplasm containing the metaphase II chromosomes using a fine sharpened pipette (17-19 μm in diameter) mounted on Narishige micromanipulators (Narishige, Tokyo, Japan). Enucleation was verified by quickly exposing the enucleated oocytes to UV light and observation using fluorescence microscopy.

Fibroblasts of median size and morphologically round smooth shape were combined with enucleated oocytes using the same pipette that was used for enucleation, then oocyte-fibroblast couplets were returned to TCM199+10% FCS until electrocell fusion. Electrofusion was performed by applying two 25 μsec 2.3 kV/cm DC pulses delivered by a BTX Electrocell Manipulator 200 (BTX, San Diego, Calif.) within 24 hpm. All couplets were then moved to TCM 199 supplemented with 10 % FCS and 5.0 μg/ml cytochalasin B (Sigma) and cultured for 1 hr. Fusion was evaluated after additional 1 hr cultured in TCM 199 supplemented with 10% FCS and then only successfully fused cybrids were selected and for activation treatment.

Activation of Electrofused Nuclear Transfer Embryos

Activation was performed 2 hours after fusion by a 4 minute incubation in 5 μM ionomycin (Calbiotechem; San Diego, Calif.) followed by 4 minutes in TL-Hepes+30 mg/ml BSA then washed twice in TL-Hepes+3 mg/ml BSA. Fusion was assessed at this time by light microscopy. Successfully fused embryos were then plated in a 500 μL Nunc 4-well containing M199+10% fetal bovine serum containing 10 μg/ml cycloheximide(Sigma C-7698)+5 μg/ml cytochalasin B(Sigma C-6762) and incubated for 5 hours.

In/Vitro Culture After Fusion

Embryos were cultured in G1G2 media for 7 days as described previously (Gardner et al BOR 1994). The formulation for G1G2 media is as follows: sodium chloride(NaCl), potassium chloride(KCl), sodium di-hydrogen phosphate(NaH2PO4), sodium bicarbonate(NaHCO3), Hepes, calcium chloride(CaCl2), magnesium sulfate(MgSO4), MOPS(buffer just like Hepes from Dom): 3-N-morpholino-propanesulfonic acid, 8 mg/ml BSA (pentex® BSA Serological Proteins, Inc. Kankakee, Ill.), glucose, sodium lactate, sodium pyruvate, taurine, alanyl-glutamine, essential AA, non-essential AA, EDTA, Vitamins, Penicillin G, Phenol Red, Xtreme™ water (pH: G1.2™ 7.3±0.10; G2.2™ 7.3±0.10; osmorality: G1.2™ 255±6; G2.2™ 260±6). Embryo development was assessed 7 days after cell fusion by stage of morphological development and cell number.

The resulting cloned embryos were periodically removed from the incubator to monitor development. Embryos developing normally as judged by cleavage division were either fixed and stained for cell counts or transferred into the reproductive tract of cows for in vivo development to term.

Embryo Transfer and Pregnancy Diagnosis

NT blastocysts were classified as to their morphology on Day 7 and only two hatching or expanded blastocysts were non-surgically transferred when synchronized recipients were available. Pregnancy status was assessed by transrectal ultrasonography (Aloka 500, 5-MHz transducer; Aloka Co., Tokyo, Japan) at 40 days after nuclear transfer and carefully rechecked regularly.

Statistics

Statistical comparisons between treatment groups were carried out using the Chi-square test, Student's t-test, and ANOVA with Fisher's exact value. All comparisons were performed using the Statview® software(SAS Institute, Cary, N.C.). Experiments were repeated at least three different times, and the number of oocytes used per experiment is indicated in Table 1.

TABLE I
Periodical Report of Bull # 86 Cloning Project

| | #enu- | #recom- | | | n = 20 | | | n = 14 | |
|---|---|---|---|---|---|---|---|---|---|
| n = 26 | cleated | bined | #fused | % of fused | #cultured | #cleaved | % cleaved | Total BL | % BL(fused) |
| Jan. 27, 2000 | 72 | 72 | 46 | 63.9 | 26 | 24 | 92.3 | 12 | 46.2 |
| Feb. 1, 2000 | 58 | 58 | 49 | 84.5 | 24 | 20 | 83.3 | 10 | 41.7 |
| Feb. 8, 2000 | 62 | 62 | 43 | 69.4 | 43 | 39 | 90.7 | 23 | 53.5 |
| Feb. 15, 2000 | 62 | 62 | 47 | 75.8 | 47 | 45 | 95.7 | 19 | 40.4 |
| Feb. 17, 2000 | 79 | 79 | 69 | 87.3 | | | | | |
| Feb. 22, 2000 | 114 | 114 | 88 | 77.2 | | | | | |
| Feb. 24, 2000 | 76 | 76 | 60 | 78.9 | 30 | 28 | 93.3 | 6 | 20 |
| Mar. 2, 2000 | 57 | 57 | 45 | 78.9 | 23 | 23 | 100 | 9 | 39.1 |
| Mar. 7, 2000 | 114 | 114 | 74 | 64.9 | 25 | 24 | 96 | 8 | 32 |
| Mar. 9, 2000 | 62 | 62 | 46 | 74.2 | | | | | |
| Mar, 21, 2000 | 78 | 78 | 64 | 82.1 | 50 | 47 | 94 | 20 | 40 |
| Mar. 23, 2000 | 58 | 58 | 42 | 72.4 | 42 | 39 | 92.9 | 25 | 59.5 |
| Mar. 29, 2000 | 48 | 48 | 43 | 89.6 | 43 | 34 | 79.1 | 21 | 48.8 |
| Apr. 4, 2000 | 64 | 64 | 46 | 71.9 | 46 | 42 | 91.3 | 20 | 43.5 |
| Apr. 5, 2000 | 67 | 67 | 55 | 82.1 | 44 | 43 | 97.7 | 29 | 65.9 |
| Apr. 11, 2000 | 68 | 68 | 61 | 89.7 | 50 | 45 | 90 | 21 | 42 |
| Apr. 12, 2000 | 34 | 34 | 30 | 88.2 | 30 | 29 | 96.7 | 12 | 40 |
| Apr. 20, 2000 | 30 | 30 | 29 | 96.7 | 29 | 18 | 62.1 | | |
| Apr. 25, 2000 | 30 | 30 | 23 | 76.7 | 23 | 15 | 65.2 | | |
| Apr. 27, 2000 | 64 | 64 | 58 | 90.6 | | | | | |
| May 2, 2000 | 35 | 35 | 34 | 97.1 | 34 | 30 | 88.2 | | |
| May 4, 2000 | 35 | 35 | 30 | 85.7 | 30 | 27 | 90 | | |
| May 9, 2000 | 32 | 32 | 27 | 84.4 | 27 | 27 | 100 | | |
| May 10, 2000 | 74 | 74 | 60 | 81.1 | | | | | |
| May 16, 2000 | 46 | 46 | 33 | 71.7 | | | | | |
| May 23, 2000 | 38 | 38 | 33 | 86.8 | 33 | 33 | 100 | | |
| subtotal | 2696 | 2696 | 2113 | 80.7213 | 1192 | 1085 | 90.02491 | 458 | 47.61429 |
| | | | | 8.714352 | | | 10.19782 | | 10.80923 |

| n = 26 | | n = 5 | | | | |
|---|---|---|---|---|---|---|
| | % BL(cleaved) | ET(BL) | recipients | pregnant | preg(%) | |
| Jan. 27, 2000 | 50 | 7 | 4 | 1 | | |
| Feb. 1, 2000 | 50 | 6 | 3 | 0 | | |
| Feb. 8, 2000 | 59 | 18 | 9 | 8 | | |
| Feb. 15, 2000 | 42.2 | 2 | 1 | 0 | | |
| Feb. 17, 2000 | | | | | | contamination |
| Feb. 22, 2000 | | | | | | sperm factor |
| Feb. 24, 2000 | 21.4 | | | | | |
| Mar. 2, 2000 | 39.1 | | | | | |
| Mar. 7, 2000 | 33.3 | | | | | sperm factor |
| Mar. 9, 2000 | | | | | | contamination |
| Mar, 21, 2000 | 42.6 | | | | | |
| Mar. 23, 2000 | 64.1 | | | | | |
| Mar. 29, 2000 | 61.8 | | | | | |
| Apr. 4, 2000 | 47.6 | | | | | |
| Apr. 5, 2000 | 67.4 | | | | | |
| Apr. 11, 2000 | 46.7 | 6 | 3 | 1 | | |
| Apr. 12, 2000 | 41.4 | | | | | |
| Apr. 20, 2000 | | | | | | |
| Apr. 25, 2000 | | | | | | |
| Apr. 27, 2000 | | | | | | contamination |
| May 2, 2000 | | | | | | |
| May 4, 2000 | | | | | | |
| May 9, 2000 | | | | | | |
| May 10, 2000 | | | | | | killed by |
| | | | | | | osmorality |
| May 16, 2000 | | | | | | contamination |
| May 23, 2000 | | | | | | |
| subtotal | 39 | 20 | 10 | | | |
| | 12.60036 | | | | | |

### Example 2

Fibroblast Cultures

Alcohol cleaned ear punches were collected into 10 mls of a modified Hanks Balanced Salt Solution made as follows: to 500 ml of Hanks Balanced Salt Solution (without calcium or magnesium) add 5.0 mls of Fungizone (Gibco), 5.0 mls of Pen-Strep (Gibco), 0.5 mls of Gentamycin (0.01 mg/ml final concentration), 5.0 mls of L-glutamine (Gibco), 5.0 mls of Non-Essential Amino Acids (Gibco), 5.0 mls of MEM Vitamins (Gibco), 5.0 mls of Sodium Pyruvate (Gibco). The media was then filter sterilized.

Each ear punch was washed in 5 petri dishes containing 5 mls each of above solution. (Tissue in the first petri dish was shaved of hair with a scalpel blade.) At this point the tissue was placed in a sterile tube containing 5 mIs of filter sterilized DMEM (to a 500 ml bottle of DMEM add 50 mls of Heat-inactivated (56° for 30 minutes) Hyclone Fetal Calf Serum, 5.5 mls of Fungizone (Gibco), 5.5 mls of Pen-Strep (Gibco), 0.6 mls of Gentamycin (0.01 mg/ml final concentration)), 5.5 mls of L-glutamine (Gibco), 5.5 mls of Non-Essential Amino Acids (Gibco), 5.5 mls of MEM Vitamins (Gibco), 5.5 mls of Sodium Pyruvate (Gibco)) and refrigerated overnight prior to use (although an overnight incubation is not required).

The next day, a slice of the skin was placed in a sixth petri dish containing 5 mls of the DMEM with additives. The slice was cut it into 20 to 30 small sections. These sections were transferred to 3 small tissue culture flasks with enough media to barely cover the bottom of the flasks. Sections were transferred by pipette into a flask for culture. The flask was scored underneath each piece to assist with adherence and the sections incubated overnight at 37° C. in 5% CO₂ and moisture.

The flask was periodically checked microscopically for fungal or bacterial contamination. Periodically, media was removed from all flasks and replace with just enough media to cover bottom. The flasks were examined daily and the media changed every two days. Once sections adhered, 4 mls of media was added to each flasks. Sections were maintained in continuous culture for 2 ½ to 3 weeks, at which time the fibroblasts were confluent.

When confluent, cultures were cryopreserved in liquid nitrogen. This procedure takes about 3 ½ to 4 weeks from start to finish. It was determined that cells will only survive being divided 20-30 times, so freezing was generally carried out after a minimum number of passes.

To divide cultures Gibco dehydrated Trypsin-EDTA was used to remove the adherent population. All media from each flask was removed and the cells rinsed with 2-4 mls of Hanks. 0.6 mls of 1× Trypsin-EDTA was added and allowed to spread over the cells. The cells are then incubated for 5 to 15 minutes, with periodic observation under a microscope to determine when the cells have rounded and loosened. The flask may be agitated to speed the process. As trypsin is detrimental to cells, the time and volume of trypsin was kept to a minimum. The cells were washed and the trypsin neutralized by the addition of 4 mls of DMEM with serum and additives. The cells were then transferred to a polypropylene centrifuge tube and centrifuge at 1200 rpms for five minutes. The cells were subsequently resuspended cells in fresh culture media and subdivided in flasks. At this time, cells were also frozen. Cells to be frozen were resuspended in cold culture medium with 10% DMSO and placed into freezing vials on ice. The cells were then frozen at −70° C. in liquid nitrogen vapor overnight and then transferred to liquid nitrogen for long term storage.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents, which are both chemically and physiologically related, may be substituted for the agents described herein while the same or similar results would be achieved. All such similar substitutes and modifications apparent to those skilled in the art are deemed to be within the spirit, scope and concept of the invention as defined by the appended claims.

### References

The following references, to the extent that they provide exemplary procedural or other details supplementary to those set forth herein, are specifically incorporated herein by reference.

U.S. Pat. No. 4,683,194

U.S. Pat. No. 4,683,195

U.S. Pat. No. 4,683,202

U.S. Pat. No. 4,994,384

U.S. Pat. No. 5,057,420

U.S. Pat. No. 5,292,639

U.S. Pat. No. 5,580,729

U.S. Pat. No. 5,891,617

U.S. Pat. No. 5,945,577

U.S. Pat. No. 5,964,096

U.S. Pat. No. 6,011,197

U.S. Pat. No. 6,107,543

U.S. Pat. No. 6,114,118

U.S. Pat. No. 6,147,276

EP0930009A1

PCT/US92/00599

WO 90/03432

WO 94/24274

WO 94/26884

WO 99/01164A1

WO 99/34669A1

WO 99/37143A2

Abir et al., J. Asst. Reprod. Gen., 15:469-477 (1998).

Adams et al, Rev. Sci. Tech. Off. Int. Epiz., 17:200-219 (1998).

Adams et al, Arch. Tierz. 42:42-55 (1999).

Adams, L. G., Development of Live Brucella Vaccines. In Advances in Brucellosis Research, Texas A&M University Press, College Station, Tex., pp. 250-270 (1990).

Barthel et al, Amer. J. Vet. Res. 61:1140-1144 (2000).

Bavister et al., Biol Reprod.;28(1):235-47, (1983).

Blackwell et al., Immunol. Lett. 43, 99-107 (1994).

Blackwell et al., Mol. Med. 1(2):194-205 (1995).

Bolamba et al., Theriogenology 49:933-942 (1998).

Breider et al, Am. J. Vet. Res., 48:1746-1750 (1987).

Campbell et al, Nature, 380:64-66 (1996).

Campbell et al, Vet. Immunol. Immunopathol., 34:291-305 (1992).

Campbell et al, Vet. Immunol. Immunopathol., 41 :295-306 (1994).

Cellier et al., J Exp. Med. 180(5):1741-52 (1994).

Collas et al, Mol. Reprod. Dev. 38(3):264-7 (1994).

Davis et al, J. Wildl. Dis. 26(3):360-371 (1990).

de Kanter et al. Xenobiotica, 28(3):225-34 (1998).

Dietrich et al. Economic and Epidemiologic Analysis of U.S. Bovine Brucellosis Programs. Primary Report, Vol. I, p. 1-24. Texas A&M University, College Station, Tex. (1986).

Dominko et al. Biol. Reprod., 62(1):150-4 (2000)

Elsheikh et al. J. Vet. Med. Sci., 59(2):107-13 (1997).

Essey, “Bovine Tuberculosis Eradication: A National Challenge” In Proc. of the 12th Annual World Assoc. of Vet. Microbiologists, Immunologits, and Specialists in Infectious Diseases, University of California, David C A B. Osburn (Ed.) p. 38-46.

Essey et al., Vet Microbiol 40(1-2):15-22 (1994).

Estes et al, Animal Genetics, 21:181-190 (1990).

Estes et al, Vet. Immunol. & Immunopathol., 25:61-72 (1990).

Feng et al, Genome Research 6:956-964 (1996).

Fulka et al,. Int. J. Dev. Biol., 37(3): 433-9 (1993).

Fulka et al. Mol. Reprod. Dev., 34(4): 427-30 (1993).

Gianaroli et al., Hum. Reprod. 14(4):1034-8 (1999).

Harmon et al, Macrophage function and immune response of Brucella abortus naturally resistant and susceptible cattle. In: Genetic Control of Host Resistance to Infection and Malignancy, E. Skamene ed., New York: Alan R. Lis, Inc., pp 345-354 (1985).

Harmon et al, Am. J. Vet. Res., 48:119-125 (1987).

Harmon et al, Am. J. Vet. Res., 49:1092-1097 (1988).

Harmon et al, Am. J. Vet. Res., 50(4):459-465 (1989).

Hewitt et al., Animal Reproduction Science, 55:63-75 (1999).

Hewitt et al., Theriogenology 49:1083-1101 (1998).

Hill et al., Biol. Reprod., 62(5):1135-40 (2000).

Horin et al, European Journal of Immunogenetics 26:311-313 (1999).

Ivanyi, Parasitol. Today 10,416-417 (1994).

Karnikova et al. Reprod. Nutr. Dev., 38(6):643-51 (1998).

Kato et al., Science, 282:2095-2098 (1998).

Keefer et al, Biol. Reprod, 50(4):935-9 (1994).

Koehler et al., Genetics 129(1):247-55 (1991).

Kraemer et al, Theriogenology, 11:51-62 (1979).

Kraemer et al., Embryo transfer in dogs and cats. Proceedings of the 2nd International Congress on Embryo Transfer and In Vitro Fertilization, Annecy, France. Eds. Meriut, C H and Bonneau, M. (1982)

Lindberg et al., Biochem. Genet., 30:27 (1992).

Liu et al., Science in China, 40:152-158, (1998).

Martin et al., Livestock disease eradication: Evaluation of the cooperative state-federal bovine tuberculosis eradication program. P. 1-97. National Research Council, Washington, D.C. (1994).

McDonald, Reproduction patterns of dogs. In Veterinary Endocrinology and Reproduction. pp 431-445. Lea and Febiger, Philadelphia (1980).

Mullis, Ann. Biol. Clin. (Paris), 48(8):579-82 (1990).

Nathan, Cell 82:873-876(1995).

Nickson et al., Journal of Reproduction and Fertility 47:231-240 (1993).

Oktay et al., Fertility and Sterility, 69:1-7 (1998).

Pierson et al., HACCP: Principles and Applications. AVI Van Nostrand Reinhold, New York, N.Y., 212 (1992).

Price et al, Infect. Immun., 58:879-886 (1990).

Price et al, Vet. Immunol. Immunopathol., 26:353-365 (1990).

Price et al, Vet. Immunol. Immunopathol., 36:265-279 (1993).

Qureshi et al, Vet. Immunol. Immunopath., 50:55-66 (1996).

Renard et al., The Lancet, 353:1489-1491 (1998).

Sheridan et al., Burns, 24:46-48 (1998).

Simione, F. P. Cryopreservation Manual, Nalge Nunc Internation Corporation, Rochester, New York (1998).

Sims et al., Proc. Natl. Acad. Sci., 90:6143-6147 (1993).

Smith et al, Am. J. Vet. Res., 51(4):512-517 (1990).

Smith et al, Am. J. Vet. Res., 51(4):518-523 (1990).

Smith et al, Vet. Immunol. Immunopathol., 25:83-97 (1990).

Smith et al, Vet. Immunol. Immunopathol., 26:31-48 (1990).

Sommer et al., Exp. Toxic Pathol. 51:229-234 (1999).

Tatham et al., Biol. Reprod, 53(5):1088-94 (1995).

Templeton et al, Natural Resistance to Bovine Brucellosis. In Advances in Brucellosis Research, Texas A&M University Press, College Station, Tex. pp. 144-150 (1990).

Templeton et al, JAVMA, 192(9):1306-1314 (1988).

Vanderzant, Subcommittee on Microbiological Criteria, Committee on Food Production, Food and Nutrition Board, National Research Council, An Evaluation of the Role of Microbiological Criteria for Foods and Food Ingredients. National Academy Press, Washington, D.C. 436 (1985).

Varner et al., Animal Welfare, 8:407-420 (1999).

Vidal et al., Cell 73, 469-485 (1993).

Vidal et al., J. Leukoc Biol. 58(4):382-90 (1995).

Wakayama et al., Nature, 394:369-374 (1998).

Wakayama, Nat. Genet., 22:127-128 (1999).

Wells et al., Cells Biol. Reprod., 60:996-1005 (1999).

Wilmut et al., Nature, 385:810-813 (1997).

Wusteman et al., Curr. Eye Res., 19(3):228-233, (1999).

Yamada et al., J. Reprod. Fert., Suppl., 47:227-229 (1993).

Zieger et al., Cell Transplant, 8(3):277-84 (1999).

What is claimed is:

1. A method of cloning a bovine comprising:
(a) contacting an enucleated oocyte with a composition comprising nuclear DNA from a cryopreserved bovine somatic cell;
(b) fusing the oocyte and the composition to form a cybrid; and
(c) transferring the cybrid into the reproductive tract of a cow.

2. The method of claim 1, wherein the cell has be cryopreserved for at least 1 year.

3. The method of claim 2, wherein the cell has been cryopreserved for at least 5 years.

4. The method of claim 2, wherein the cell has been cryopreserved for at least 10 years.

5. The method of claim 3, wherein the cell has been cryopreserved for at least 15 years.

6. The method of claim 1, wherein the composition comprises a nucleus.

7. The method of claim 6, wherein the nucleus is comprised in a cryopreserved bovine somatic cell.

8. The method of claim 1, wherein the cybrid undergoes cell division prior to transferring.

9. The method of claim 8, wherein the cybrid undergoes at least two cell divisions.

10. The method of claim 1, wherein the transferred cybrid develops into a viable bovine.

11. The method of claim 1, wherein the oocyte is a bovine oocyte.

12. The method of claim 11, wherein the oocyte and the nuclear DNA are from the same bovine species.

13. The method of claim 12, wherein the oocyte and nuclear DNA are from the same bovine breed.

14. The method of claim 13, wherein the oocyte and nuclear DNA are from the same animal.

15. The method of claim 1, wherein the oocyte is in metaphase II.

16. The method of claim 1, wherein the oocyte is cultured in vitro prior to enucleation.

17. The method of claim 1, further comprising activating the cybrid.

18. The method of claim 17, wherein activating comprises applying an electrical pulse.

19. The method of claim 17, wherein activating comprises applying a chemical treatment.

20. The method of claim 1, wherein fusing the oocyte and the composition comprises applying an electrical pulse.

21. The method of claim 1, wherein fusing the oocyte and the composition comprises injecting the composition into the oocyte.

22. The method of claim 21, wherein the oocyte and the composition are incubated in media comprising fusion media.

23. The method of claim 1, wherein the cybrid is cultured in vitro for at least 24 hours prior to transfer.

24. The method of claim 1, wherein the cybrid is cultured in vitro until at least the 4-8 cell stage.

25. The method of claim 1, wherein the cybrid is frozen and thawed prior to transfer into the cow.

26. The method of claim 1, wherein the estrous cycle of the cow has been synchronized with the estrous cycle of the oocyte donor.

27. The method of claim 1, wherein the estrous cycle of the cow has been synchronized with the developmental stage of the cybrid.

28. The method of claim 1, wherein the nuclear DNA comprises a heterologous DNA sequence.

29. A bovine whose nuclear genome is identical to a single parent, wherein said single parent comprises a polymorphic NRAMP1 gene that confers disease resistance.

30. The bovine of claim 29, wherein said disease resistance is to disease caused by an intracellular pathogen.

31. The bovine of claim 30, wherein said intracellular pathogen is brucellosis, tuberculosis, paratuberculosis or salmonellosis

32. The bovine of claim 29, wherein said polymorphic NRAMP1 gene exhibits polymorphism in the 3′ UTR.

33. The bovine of claim 32, wherein said polymorphism in the 3′ UTR is at nucleotide 1782 of SEQ ID NO. 1.

34. The bovine of claim 32, wherein said polymorphism in the 3′ UTR is at nucleotide 1789 of SEQ ID NO. 3.

35. The bovine of claim 29, wherein the parent is an ATCC# MXXM cell.

36. A bovine whose nuclear genome is identical to a single parent produced from a method comprising:
(a) contacting an enucleated oocyte with a composition comprising nuclear DNA from a bovine somatic cell, wherein said bovine somatic cell is derived from a cell line comprising a polymorphic NRAMP1 gene;
(b) fusing the oocyte and the composition to form a cybrid; and
(c) transferring the cybrid into the reproductive tract of a cow.

37. The bovine of claim 36, wherein the bovine comprises a polymorphic NRAMP1 gene that confers disease resistance.

38. The bovine of claim 36, wherein the disease resistance is to diseases caused by an intracellular pathogen.

39. The bovine of claim 38, wherein the intracellular pathogen is brucellosis, tuberculosis, paratuberculosis, or salmonellosis.

40. The bovine of claim 36, wherein the polymorphic NRAMP1 gene exhibits polymorphism in the 3′ UTR.

41. The bovine of claim 40, wherein the polymorphism in the 3′ UTR is at nucleotide 1782 of SEQ ID NO. 3.

42. The bovine of claim 40, wherein the polymorphism in the 3′ UTR is at nucleotide 1789 of SEQ ID NO. 3.

43. The bovine of claim 36, wherein the single parent is an ATCC# MXXM cell and the cell line is ATCC# MXXM.

44. The bovine of claim 36, wherein the oocyte and the nucleus are from the same bovine species.

45. The bovine of claim 36, wherein the composition comprises a nucleus.

46. The bovine of claim 45, wherein the nucleus is comprised in a bovine somatic cell.

47. The bovine of claim 46, wherein the cybrid undergoes cell division prior to transferring.

48. The bovine of claim 47, wherein the cybrid undergoes at least two cell divisions.

49. The bovine of claim 36, wherein the oocyte is a bovine oocyte.

50. The bovine of claim 49, wherein the oocyte and nuclear DNA are from the same breed.

51. The bovine of claim 50, wherein the oocyte and nuclear DNA are from the same animal.

52. The bovine of claim 36, wherein the oocyte is in metaphase II.

53. The bovine of claim 36, wherein the oocyte is cultured in vitro prior to enucleation.

54. The bovine of claim 36, further comprising activating the cybrid.

55. The bovine of claim 54, wherein activating comprises applying an electrical pulse.

56. The bovine of claim 36, wherein fusing the oocyte and the composition comprises applying an electrical pulse.

57. The bovine of claim 36, wherein fusing the oocyte and the composition comprises injecting the composition into the oocyte.

58. The bovine of claim 57, wherein the oocyte and the composition are incubated in media comprising fusion media.

59. The bovine of claim 36, wherein the cybrid is cultured in vitro for at least 2 hours prior to transfer.

60. The bovine of claim 36, wherein the cybrid is cultured in vitro until it has undergone at least one cell division.

61. The bovine of claim 36, wherein the cybrid is cultured in vitro until it has undergone cell division more than two times.

62. The bovine of claim 36, wherein the estrous cycle of the cow has been synchronized with the estrous cycle of the oocyte donor.

63. The bovine of claim 36, wherein the estrous cycle of the cow has been synchronized with the developmental stage of the cybrid.

64. A progeny bovine whose nuclear genome is identical to a single ancestor produced by mating the bovine of claim 29 or 36 with a second bovine.

65. The progeny bovine of claim 64, wherein the progeny bovine comprises a polymorphic NRAMP1 gene that confers disease resistance

66. A progeny bovine whose nuclear genome is identical to a single ancestor and comprises a polymorphic NRAMP1 gene that confers disease resistance to brucellosis, tuberculosis, paratuberculosis or salmonellosis, wherein the progeny bovine is produced by mating the bovine of claim 29 or 36 with a second bovine.

67. A bovine whose nuclear genome is identical to a single ancestor and comprises a polymorphic NRAMP1 gene that confers disease resistance to brucellosis, tuberculosis, paratuberculosis or salmonellosis, wherein the bovine is produced from a method comprising:
(a) contacting an enucleated oocyte with a composition comprising nuclear DNA from a bovine somatic cell, wherein said bovine somatic cell comprises the sequence of SEQ ID NO. 3;
(b) fusing the oocyte and the composition to form a cybrid; and
(c) transferring the cybrid into the reproductive tract of a cow.
